(19) United States
(12) Patent Application Publication
(10) Pub. No.: US 2008/0072663 A1
(43) Pub. Date: Mar. 27, 2008

(54) ACCURATE AND TIMELY BODY FLUID ANALYSIS

(75) Inventors: Richard Keenan, Livermore, CA (US); Jeffrey Chiou, Union City, CA (US); Roger Tong, Berkeley, CA (US); Michael Recknor, Oakland, CA (US); Ken I. Li, Piedmont, CA (US); James R. Braig, Piedmont, CA (US); David N. Callicoat, Alameda, CA (US); Jennifer H. Gable, Newark, CA (US)

Correspondence Address: KNOBBE MARTENS OLSON & BEAR LLP, 2040 MAIN STREET, FOURTEENTH FLOOR, IRVINE, CA, 92614, US

(73) Assignee: OptiScan Biomedical Corporation, Hayward, CA (US)

(21) Appl. No.: 11/839,487

(22) Filed: Aug. 15, 2007

Related U.S. Application Data
(60) Provisional application No. 60/837,832, filed on Aug. 15, 2006.
(60) Provisional application No. 60/837,746, filed on Aug. 15, 2006.
(60) Provisional application No. 60/939,036, filed on May 18, 2007.
(60) Provisional application No. 60/939,023, filed on May 18, 2007.
(60) Provisional application No. 60/950,093, filed on Jul. 16, 2007.
(60) Provisional application No. 60/953,454, filed on Aug. 1, 2007.
(60) Provisional application No. 60/901,474, filed on Feb. 15, 2007.

(51) Int. Cl. G01N 33/00; B01J 19/00; B04B 5/10

(52) U.S. Cl. 73/61.41; 422/68.1; 422/72

(57) ABSTRACT

A method of extracting and analyzing bodily fluids from a patient at the point of care for the patient is provided. The method comprises establishing fluid communication between an analyte detection system and a bodily fluid in the patient. A portion of the bodily fluid is drawn from the patient. A first component of the bodily fluid is separated from the drawn portion, while the analyte detection system remains in fluid communication with the patient. The analyte detection system analyzes the first component to measure a concentration of an analyte in an accurate and timely manner.

### PRIORITY INFORMATION

This application claims the benefit under 35 U.S.C. § 119(e) of the following U.S. Provisional Patent Application Nos.: 60/837,832, filed Aug. 15, 2006 (attorney docket no. 171PR); 60/837,746, filed Aug. 15, 2006 (attorney docket no. 172PR); 60/901,474, filed Feb. 15, 2007 (attorney docket no. 178PR); 60/939,036, filed May 18, 2007 (attorney docket no. 183PR); 60/939,023, filed May 18, 2007 (attorney docket no. 184PR); 60/950,093, filed Jul. 16, 2007 (attorney docket no. 186PR); and 60/953,454, filed Aug. 1, 2007 (attorney docket no. 190PR). The entirety of each of the above-referenced applications is hereby incorporated by reference and made part of this specification.

### BACKGROUND

1. Field

Certain embodiments disclosed herein relate to methods and apparatus for determining the concentration of an analyte in a sample, such as an analyte in a sample of bodily fluid, as well as methods and apparatus which can be used to support the making of such determinations.

2. Description of the Related Art

It is a common practice to measure the levels of certain analytes, such as glucose, in a bodily fluid, such as blood. Often this is done in a hospital or clinical setting when there is a risk that the levels of certain analytes may move outside a desired range, which in turn can jeopardize the health of a patient. Certain currently known systems for analyte monitoring in a hospital or clinical setting suffer from various drawbacks.

### SUMMARY

In some embodiments, a method of extracting and analyzing bodily fluids from a patient at the point of care for the patient is provided. The method comprises establishing fluid communication between an analyte detection system and a bodily fluid in the patient. A portion of the bodily fluid is drawn from the patient. A first component of the bodily fluid is separated from the drawn portion, while the analyte detection system remains in fluid communication with the patient. The analyte detection system analyzes the first component to measure a concentration of an analyte.

In some embodiments, a method of preparing for analysis a bodily fluid of a patient is provided. The method comprises operably connecting a fluid separation system to the patient. A portion of the bodily fluid is draw from the patient and into the fluid separation system. A first component is separated from the drawn portion of bodily fluid with the fluid separation system, while the fluid separation system remains operably connected to the patient.

In some embodiments, a method of extracting and analyzing a bodily fluid of a patient is provided. The method comprises attaching an analyte detection system to a patient wherein the analyte detection system further comprises a fluid handling system. The fluid handling system is attached to the patient. A sample of bodily fluid is drawn from the patient into the fluid handling system. The sample is directly analyzed with the analyte detection system to measure a concentration of an analyte.

Certain objects and advantages of the invention(s) are described herein. Of course, it is to be understood that not necessarily all such objects or advantages may be achieved in accordance with any particular embodiment. Thus, for example, those skilled in the art will recognize that the invention(s) may be embodied or carried out in a manner that achieves or optimizes one advantage or group of advantages as taught herein without necessarily achieving other objects or advantages as may be taught or suggested herein.

Certain embodiments are summarized above. However, despite the foregoing discussion of certain embodiments, only the appended claims (and not the present summary) are intended to define the invention(s). The summarized embodiments, and other embodiments, will become readily apparent to those skilled in the art from the following detailed description of the preferred embodiments having reference to the attached FIGS., the invention(s) not being limited to any particular embodiment(s) disclosed.

### BRIEF DESCRIPTION OF THE DRAWINGS

The following drawings and the associated descriptions are provided to illustrate embodiments of the present disclosure and do not limit the scope of the claims.

FIG. 1 shows an embodiment of an apparatus for withdrawing and analyzing fluid samples;

FIG. 2 illustrates how various other devices can be supported on or near an embodiment of apparatus illustrated in FIG. 1;

FIG. 3 illustrates an embodiment of the apparatus in FIG. 1 connected to a patient;

FIG. 4 is a block diagram of an embodiment of a system for withdrawing and analyzing fluid samples;

FIG. 5A schematically illustrates an embodiment of a fluid system that can be part of a system for withdrawing and analyzing fluid samples;

FIG. 5B schematically illustrates another embodiment of a fluid system that can be part of a system for withdrawing and analyzing fluid samples;

FIG. 6 is an oblique schematic depiction of an embodiment of a monitoring device;

FIG. 7A shows a cut-away side view of an embodiment of a monitoring device;

FIG. 7B shows a cut-away perspective view of an embodiment of a monitoring device;

FIG. 8A illustrates an embodiment of a removable cartridge that can interface with a monitoring device;

FIG. 8B illustrates an embodiment of a fluid routing card that can be part of the removable cartridge of FIG. 8A;

FIG. 9A illustrates how non-disposable actuators can interface with the fluid routing card of FIG. 8B.

FIG. 9B illustrates a modular pump actuator connected to a syringe housing that can form a portion of a removable cartridge.

FIG. 9C shows a rear perspective view of internal scaffolding and some pinch valve pump bodies.

FIG. 10A shows an underneath perspective view of a sample cell holder attached to a centrifuge interface, with a view of an interface with a sample injector.

FIG. 10B shows a plan view of a sample cell holder with hidden and/or non-surface portions illustrated using dashed lines.

FIG. 10C shows a top perspective view of the centrifuge interface connected to the sample holder.

FIG. 11A shows a perspective view of an example optical system.

FIG. 11B shows a filter wheel that can be part of the optical system of FIG. 11A.

FIG. 12 schematically illustrates an embodiment of an optical system that comprises a spectroscopic analyzer adapted to measure spectra of a fluid sample;

FIG. 13 is a flowchart that schematically illustrates an embodiment of a method for estimating the concentration of an analyte in the presence of interferents;

FIG. 14 is a flowchart that schematically illustrates an embodiment of a method for performing a statistical comparison of the absorption spectrum of a sample with the spectrum of a sample population and combinations of individual library interferent spectra;

FIG. 15 is a flowchart that schematically illustrates an example embodiment of a method for estimating analyte concentration in the presence of the possible interferents;

FIGS. 16A and 16B schematically illustrate the visual appearance of embodiments of a user interface for a system for withdrawing and analyzing fluid samples;

FIG. 17 schematically depicts various components and/or aspects of a patient monitoring system and the relationships among the components and/or aspects;

FIG. 18 is a chart depicting measurement results;

FIG. 19 is a graph showing measurement results;

FIG. 20 is a graph showing measurement results;

FIG. 21 is a graph showing measurement results;

FIG. 22 is a graph showing measurement results;

FIG. 23 is a graph showing measurement results;

FIG. 24 is a graph showing the results of a simulation;

FIG. 25 is a graph showing the results of a simulation;

FIG. 26 is a graph showing the results of a simulation; and

FIG. 27 is a bar chart showing the elapsed time during a measurement cycle.

Reference symbols are used in the figures to indicate certain components, aspects or features shown therein, with reference symbols common to more than one figure indicating like components, aspects or features shown therein.

### DETAILED DESCRIPTION OF THE PREFERRED EMBODIMENTS

Although certain preferred embodiments and examples are disclosed below, inventive subject matter extends beyond the specifically disclosed embodiments to other alternative embodiments and/or uses of the invention, and to modifications and equivalents thereof. Thus, the scope of the inventions herein disclosed is not limited by any of the particular embodiments described below. For example, in any method or process disclosed herein, the acts or operations of the method or process may be performed in any suitable sequence and are not necessarily limited to any particular disclosed sequence. For purposes of contrasting various embodiments with the prior art, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient.

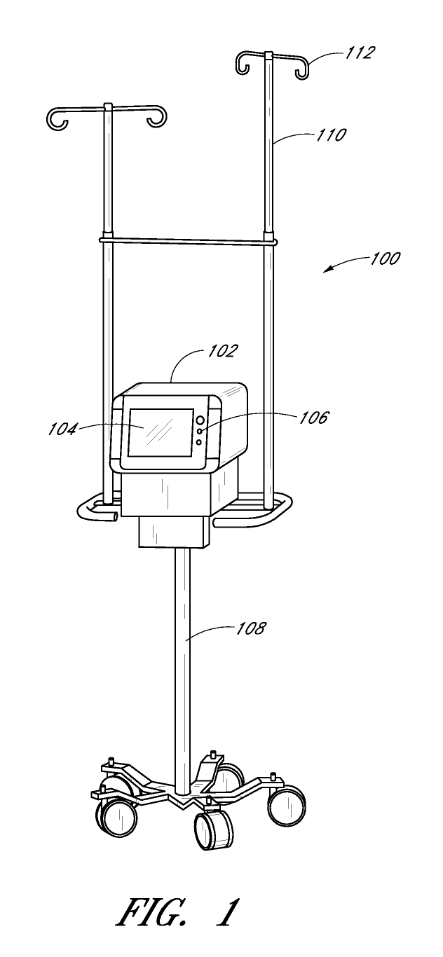

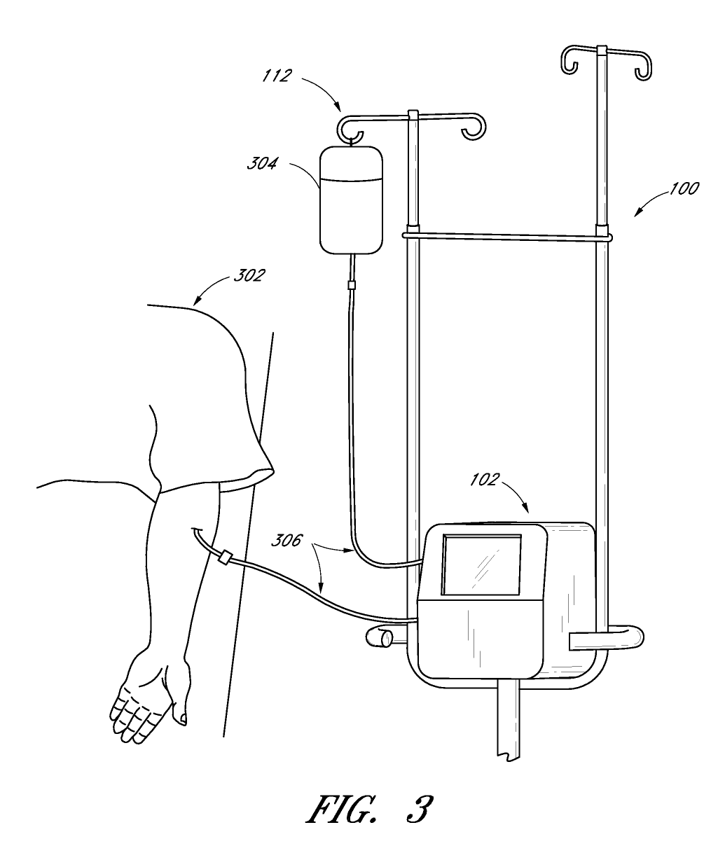

FIG. 1 shows an embodiment of an apparatus 100 for withdrawing and analyzing fluid samples. The apparatus 100 includes a monitoring device 102. In some embodiments, the monitoring device 102 can be an “OptiScanner®,” available from OptiScan Biomedical Corporation of Hayward, Calif. In some embodiments, the device 102 can measure one or more physiological parameters, such as the concentration of one or more substance(s) in a sample fluid. The sample fluid can be, for example, whole blood from a patient 302 (see, e.g., FIG. 3). In some embodiments, the device 100 can also deliver an infusion fluid to the patient 302.

In the illustrated embodiment, the monitoring device 102 includes a display 104 such as, for example, a touch-sensitive liquid crystal display. The display 104 can provide an interface that includes alerts, indicators, charts, and/or soft buttons. The device 102 also can include one or more inputs and/or outputs 106 that provide connectivity.

In the embodiment shown in FIG. 1, the device 102 is mounted on a stand 108. The stand 108 can be easily moved and includes one or more poles 110 and/or hooks 112. The poles 110 and hooks 112 can be configured to accommodate other medical devices and/or implements, including, for example, infusion pumps, saline bags, arterial pressure sensors, other monitors and medical devices, and so forth.

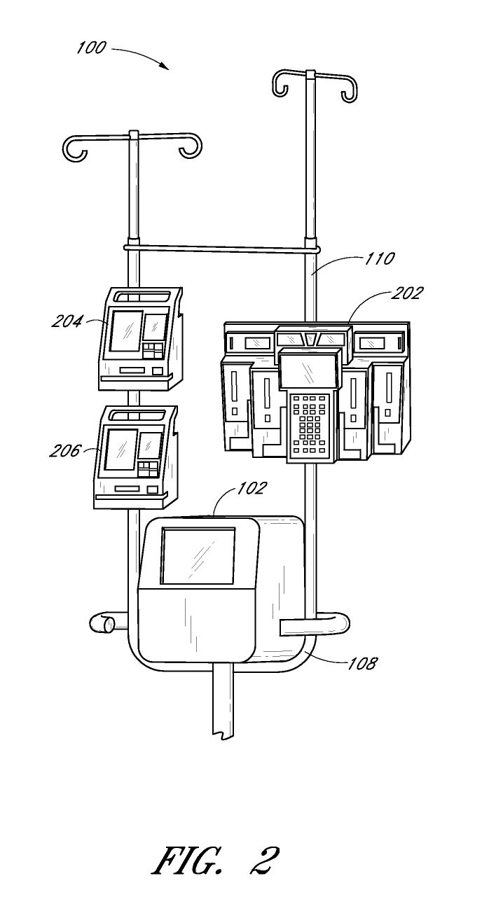

FIG. 2 illustrates how various other devices can be supported on or near the apparatus 100 illustrated in FIG. 1. For example, the poles 110 of the stand 108 can be configured (e.g., of sufficient size and strength) to accommodate multiple devices 202, 204, 206. In some embodiments, one or more COLLEAGUE® volumetric infusion pumps available from Baxter International Inc. of Deerfield, Ill. can be accommodated. In some embodiments, one or more Alaris® PC units available from Cardinal Health, Inc. of Dublin, Ohio can be accommodated. Furthermore, various other medical devices (including the two examples mentioned here), can be integrated with the disclosed monitoring device 102 such that multiple devices function in concert for the benefit of one or multiple patients without the devices interfering with each other.

FIG. 3 illustrates the apparatus 100 of FIG. 1 as it can be connected to a patient 302. The monitoring device 102 can be used to determine the concentration of one or more substances in a sample fluid. The sample fluid can come from a fluid container in a laboratory setting, or it can come from a patient 302, as illustrated here. In some preferred embodiments, the sample fluid is whole blood.

In some embodiments, the monitoring device 102 can also deliver an infusion fluid to the patient 302. An infusion fluid container 304 (e.g., a saline bag), which can contain infusion fluid (e.g., saline and/or medication), can be supported by the hook 112. The monitoring device 102 can be in fluid communication with both the container 304 and the sample fluid source (e.g., the patient 302), through tubes 306. The infusion fluid can comprise any combination of fluids and/or chemicals. Some advantageous examples include (but are not limited to): water, saline, dextrose, lactated Ringer's solution, drugs, and insulin.

The illustrated monitoring device 102 allows the infusion fluid to pass to the patient 302 and/or uses the infusion fluid itself (e.g., as a flushing fluid or a standard with known optical properties, as discussed further below). In some embodiments, the monitoring device 102 may not employ infusion fluid. The monitoring device 102 may thus draw samples without delivering any additional fluid to the patient 302. The monitoring device 102 can include, but is not limited to, fluid handling and analysis apparatuses, connectors, passageways, catheters, tubing, fluid control elements, valves, pumps, fluid sensors, pressure sensors, temperature sensors, hematocrit sensors, hemoglobin sensors, calorimetric sensors, gas (e.g., “bubble”) sensors, fluid conditioning elements, gas injectors, gas filters, blood plasma separators, and/or communication devices (e.g., wireless devices) to permit the transfer of information within the monitoring device 102 or between the monitoring device 102 and a network.

In some embodiments, one or more components of the apparatus 100 can be located at another facility, room, or other suitable remote location. One or more components of the monitoring device 102 can communicate with one or more other components of the monitoring device 102 (or with other devices) by communication interface(s) such as, but not limited to, optical interfaces, electrical interfaces, and/or wireless interfaces. These interfaces can be part of a local network, internet, wireless network, or other suitable networks.

System Overview

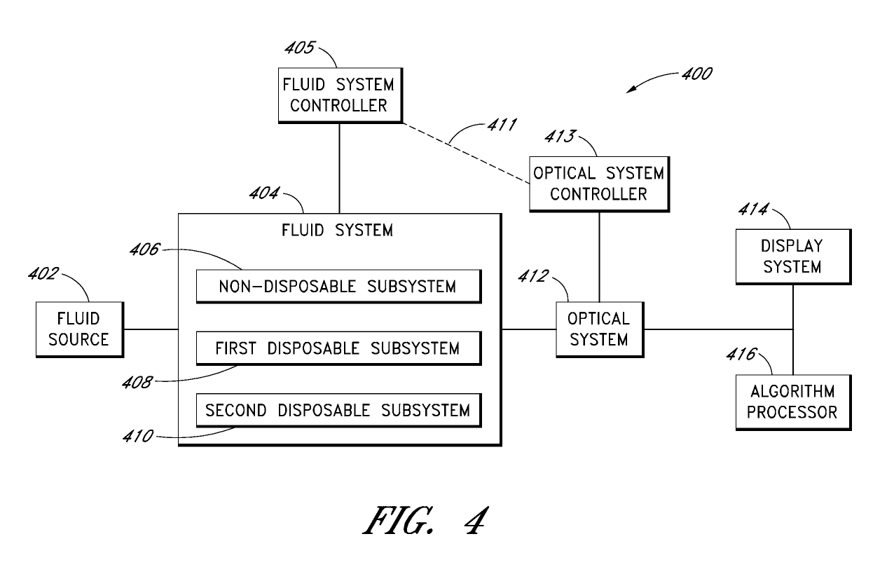

FIG. 4 is a block diagram of a system 400 for sampling and analyzing fluid samples. The monitoring device 102 can comprise such a system. The system 400 can include a fluid source 402 connected to a fluid-handling system 404. The fluid-handling system 404 includes fluid passageways and other components that direct fluid samples. Samples can be withdrawn from the fluid source 402 and analyzed by an optical system 412. The fluid-handling system 404 can be controlled by a fluid system controller 405, and the optical system 412 can be controlled by an optical system controller 413. The sampling and analysis system 400 can also include a display system 414 and an algorithm processor 416 that assist in fluid sample analysis and presentation of data.

In some embodiments, the sampling and analysis system 400 is a mobile point-of-care apparatus that monitors physiological parameters such as, for example, blood glucose concentration. Components within the system 400 that may contact fluid and/or a patient, such as tubes and connectors, can be coated with an antibacterial coating to reduce the risk of infection. Connectors between at least some components of the system 400 can include a self-sealing valve, such as a spring valve, in order to reduce the risk of contact between port openings and fluids, and to guard against fluid escaping from the system. Other components can also be included in a system for sampling and analyzing fluid in accordance with the described embodiments.

The sampling and analysis system 400 can include a fluid source 402 (or more than one fluid source) that contain(s) fluid to be sampled. The fluid-handling system 404 of the sampling and analysis system 400 is connected to, and can draw fluid from, the fluid source 402. The fluid source 402 can be, for example, a blood vessel such as a vein or an artery, a container such as a decanter, flask, beaker, tube, etc., or any other corporeal or extracorporeal fluid source. The fluid to be sampled can be, for example, blood, plasma, interstitial fluid, lymphatic fluid, or another fluid. In some embodiments, more than one fluid source can be present, and more than one fluid and/or type of fluid can be provided.

In some embodiments, the fluid-handling system 404 withdraws a sample of fluid from the fluid source 402 for analysis, centrifuges at least a portion of the sample, and prepares at least a portion of the sample for analysis by an optical sensor such as a spectrophotometer (which can be part of an optical system 412, for example). These functions can be controlled by a fluid system controller 405, which can also be integrated into the fluid-handling system 404. The fluid system controller 405 can also control the additional functions described below.

In some embodiments, at least a portion of the sample is returned to the fluid source 402. At least some of the sample, such as portions of the sample that are mixed with other materials or portions that are otherwise altered during the sampling and analysis process, or portions that, for any reason, are not to be returned to the fluid source 402, can also be placed in a waste bladder (not shown in FIG. 4). The waste bladder can be integrated into the fluid-handling system 404 or supplied by a user of the system 400. The fluid-handling system 404 can also be connected to a saline source, a detergent source, and/or an anticoagulant source, each of which can be supplied by a user, attached to the fluid-handling system 404 as additional fluid sources, and/or integrated into the fluid-handling system 404.

Components of the fluid-handling system 404 can be modularized into one or more non-disposable, disposable, and/or replaceable subsystems. In the embodiment shown in FIG. 4, components of the fluid-handling system 404 are separated into a non-disposable subsystem 406, a first disposable subsystem 408, and a second disposable subsystem 410.

The non-disposable subsystem 406 can include components that, while they may be replaceable or adjustable, do not generally require regular replacement during the useful lifetime of the system 400. In some embodiments, the non-disposable subsystem 406 of the fluid-handling system 404 includes one or more reusable valves and sensors. For example, the non-disposable subsystem 406 can include one or more pinch valves (or non-disposable portions thereof), ultrasonic bubble sensors, non-contact pressure sensors, and optical blood dilution sensors. The non-disposable subsystem 406 can also include one or more pumps (or non-disposable portions thereof). In some embodiments, the components of the non-disposable subsystem 406 are not directly exposed to fluids and/or are not readily susceptible to contamination.

The first and second disposable subsystems 408, 410 can include components that are regularly replaced under certain circumstances in order to facilitate the operation of the system 400. For example, the first disposable subsystem 408 can be replaced after a certain period of use, such as a few days, has elapsed. Replacement may be necessary, for example, when a bladder within the first disposable subsystem 408 is filled to capacity. Such replacement may mitigate fluid system performance degradation associated with and/or contamination wear on system components.

In some embodiments, the first disposable subsystem 408 includes components that may contact fluids such as patient blood, saline, flushing solutions, anticoagulants, and/or detergent solutions. For example, the first disposable subsystem 408 can include one or more tubes, fittings, cleaner pouches and/or waste bladders. The components of the first disposable subsystem 408 can be sterilized in order to decrease the risk of infection and can be configured to be easily replaceable.

In some embodiments, the second disposable subsystem 410 can be designed to be replaced under certain circumstances. For example, the second disposable subsystem 410 can be replaced when the patient being monitored by the system 400 is changed. The components of the second disposable subsystem 410 may not need replacement at the same intervals as the components of the first disposable subsystem 408. For example, the second disposable subsystem 410 can include a sample holder and/or at least some components of a centrifuge, components that may not become filled or quickly worn during operation of the system 400. Replacement of the second disposable subsystem 410 can decrease or eliminate the risk of transferring fluids from one patient to another during operation of the system 400, enhance the measurement performance of system 400, and/or reduce the risk of contamination or infection.

In some embodiments, the sample holder of the second disposable subsystem 410 receives the sample obtained from the fluid source 402 via fluid passageways of the first disposable subsystem 408. The sample holder is a container that can hold fluid for the centrifuge and can include a window to the sample for analysis by a spectrometer. In some embodiments, the sample holder includes windows that are made of a material that is substantially transparent to electromagnetic radiation in the mid-infrared range of the spectrum. For example, the sample holder windows can be made of calcium fluoride.

An injector can provide a fluid connection between the first disposable subsystem 408 and the sample holder of the second disposable subsystem 410. In some embodiments, the injector can be removed from the sample holder to allow for free spinning of the sample holder during centrifugation.

In some embodiments, the components of the sample are separated by centrifuging at a high speed for a period of time before measurements are performed by the optical system 412. For example, a blood sample can be centrifuged at 7200 RPM for 2 minutes in order to separate plasma from other blood components for analysis. Separation of a sample into the components can permit measurement of solute (e.g., glucose) concentration in plasma, for example, without interference from other blood components. This kind of post-separation measurement, (sometimes referred to as a “direct measurement”) has advantages over a solute measurement taken from whole blood because the proportions of plasma to other components need not be known or estimated in order to infer plasma glucose concentration.

An anticoagulant, such as, for example, heparin can be added to the sample before centrifugation to prevent clotting. The fluid-handling system 404 can be used with a variety of anticoagulants, including anticoagulants supplied by a hospital or other user of the monitoring system 400. A detergent solution formed by mixing detergent powder from a pouch connected to the fluid-handling system 404 with saline can be used to periodically clean residual protein and other sample remnants from one or more components of the fluid-handling system 404, such as the sample holder. Sample fluid to which anticoagulant has been added and used detergent solution can be transferred into the waste bladder.

The system 400 shown in FIG. 4 includes an optical system 412 that can measure optical properties (e.g., transmission) of a fluid sample (or a portion thereof). In some embodiments, the optical system 412 measures transmission in the mid-infrared range of the spectrum. In some embodiments, the optical system 412 includes a spectrometer that measures the transmission of broadband infrared light through a portion of a sample holder filled with fluid. The spectrometer need not come into direct contact with the sample. As used herein, the term “sample holder” is a broad term that carries its ordinary meaning as an object that can provide a place for fluid. The fluid can enter the sample holder by flowing.

In some embodiments, the optical system 412 includes a filter wheel that contains one or more filters. In some embodiments, twenty-five filters are mounted on the filter wheel. The optical system 412 includes a light source that passes light through a filter and the sample holder to a detector. In some embodiments, a stepper motor moves the filter wheel in order to position a selected filter in the path of the light. An optical encoder can also be used to finely position one or more filters.

The optical system 412 can be controlled by an optical system controller 413. The optical system controller can, in some embodiments, be integrated into the optical system 412. In some embodiments, the fluid system controller 405 and the optical system controller 413 can communicate with each other as indicated by the line 411. In some embodiments, the function of these two controllers can be integrated and a single controller can control both the fluid-handling system 404 and the optical system 412. Such an integrated control can be advantageous because the two systems are preferably integrated, and the optical system 412 is preferably configured to analyze the very same fluid handled by the fluid-handling system 404. Indeed, portions of the fluid-handling system 404 (e.g., the sample holder described above with respect to the second disposable subsystem 410 and/or at least some components of a centrifuge) can also be components of the optical system 412. Accordingly, the fluid-handling system 404 can be controlled to obtain a fluid sample for analysis by optical system 412, when the fluid sample arrives, the optical system 412 can be controlled to analyze the sample, and when the analysis is complete (or before), the fluid-handling system 404 can be controlled to return some of the sample to the fluid source 402 and/or discard some of the sample, as appropriate.

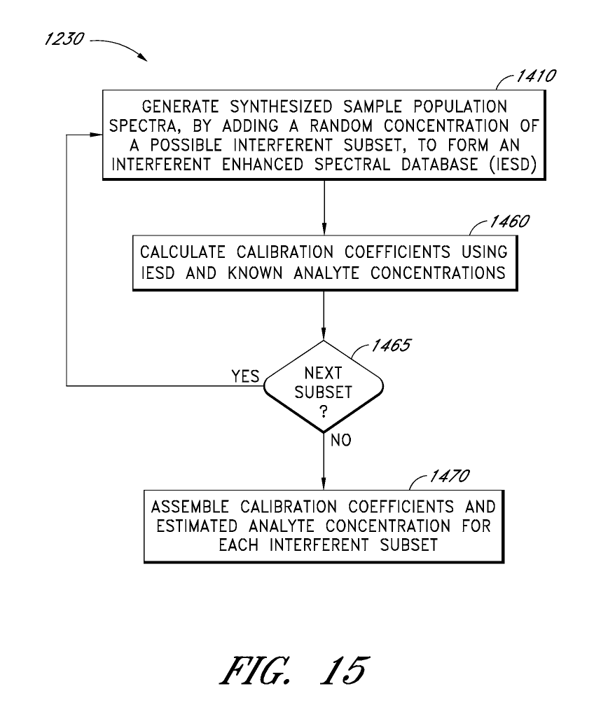

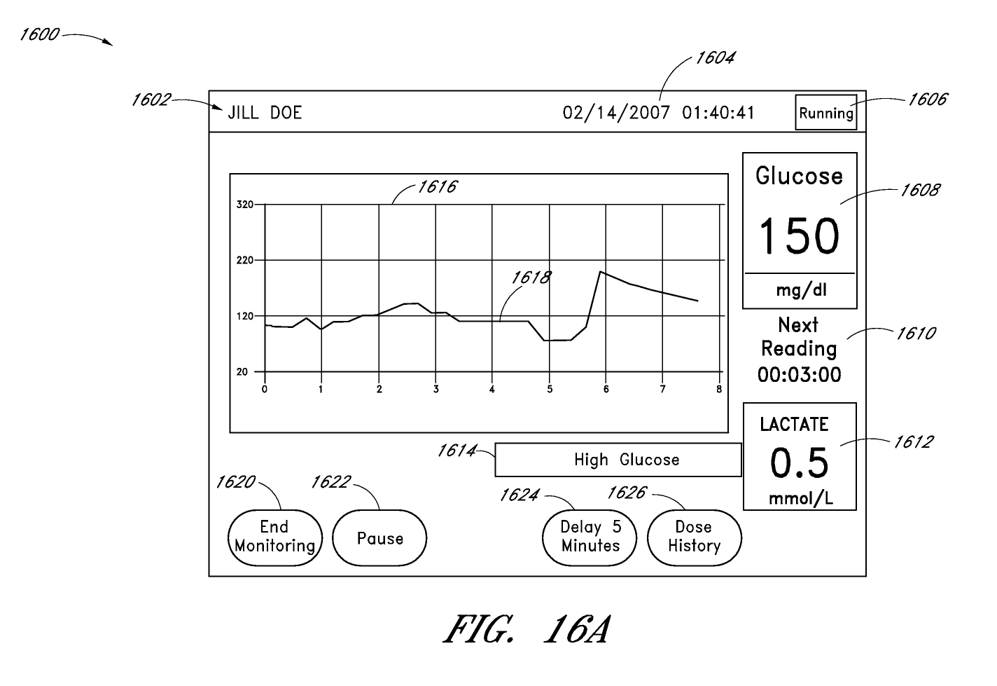

The system 400 shown in FIG. 4 includes a display system 414 that provides for communication of information to a user of the system 400. In some embodiments, the display 414 can be replaced by or supplemented with other communication devices that communicate in non-visual ways. The display system 414 can include a display processor that controls or produces an interface to communicate information to the user. The display system 414 can include a display screen. One or more parameters such as, for example, blood glucose concentration, system 400 operating parameters, and/or other operating parameters can be displayed on a monitor (not shown) associated with the system 400. An example of one way such information can be displayed is shown in FIGS. 16A and 16B. In some embodiments, the display system 414 can communicate measured physiological parameters and/or operating parameters to a computer system over a communications connection.

The system 400 shown in FIG. 4 includes an algorithm processor 416 that can receive spectral information, such as optical density (OD) values (or other analog or digital optical data) from the optical system 412 and or the optical system controller 413. In some embodiments, the algorithm processor 416 calculates one or more physiological parameters and can analyze the spectral information. Thus, for example and without limitation, a model can be used that determines, based on the spectral information, physiological parameters of fluid from the fluid source 402. The algorithm processor 416, a controller that may be part of the display system 414, and any embedded controllers within the system 400 can be connected to one another with a communications bus.

Some embodiments of the systems described herein (e.g., the system 400), as well as some embodiments of each method described herein, may include a computer program accessible to and/or executable by a processing system, e.g., a one or more processors and memories that are part of an embedded system. Indeed, the controllers may comprise one or more computers and/or may use software. Thus, as will be appreciated by those skilled in the art, embodiments of the disclosed inventions may be embodied as a method, an apparatus such as a special purpose apparatus, an apparatus such as a data processing system, or a carrier medium, e.g., a computer program product. The carrier medium carries one or more computer readable code segments for controlling a processing system to implement a method. Accordingly, various ones of the disclosed inventions may take the form of a method, an entirely hardware embodiment, an entirely software embodiment or an embodiment combining software and hardware aspects. Furthermore, any one or more of the disclosed methods (including but not limited to the disclosed methods of measurement analysis, interferent determination, and/or calibration constant generation) may be stored as one or more computer readable code segments or data compilations on a carrier medium. Any suitable computer readable carrier medium may be used including a magnetic storage device such as a diskette or a hard disk; a memory cartridge, module, card or chip (either alone or installed within a larger device); or an optical storage device such as a CD or DVD.

Fluid Handling System

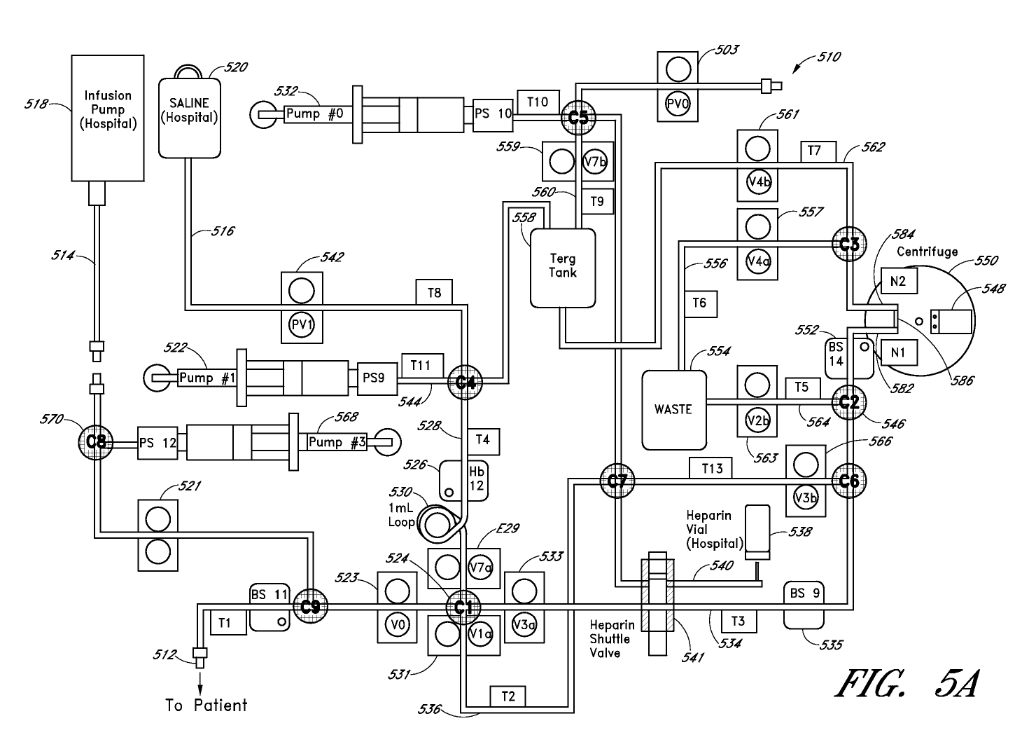

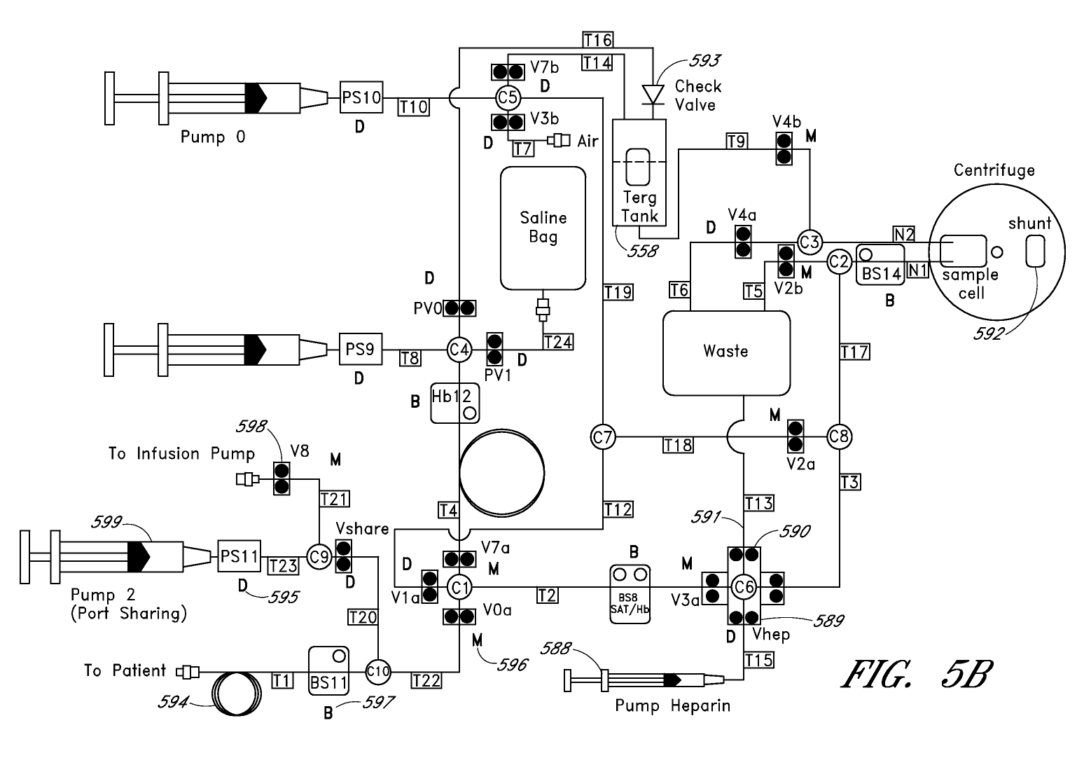

The generalized fluid-handling system 404 can have various configurations. In this context, FIG. 5A schematically illustrates the layout of an example embodiment of a fluid system 510. In this schematic representation, various components are depicted that may be part of a non-disposable subsystem 406, a first disposable subsystem 408, a second disposable subsystem 410, and/or an optical system 412. The fluid system 510 is described practically to show an example cycle as fluid is drawn and analyzed.

In addition to the reference numerals used below, the various portions of the illustrated fluid system 510 are labeled for convenience with letters to suggest their roles as follows: T# indicates a section of tubing. C# indicates a connector that joins multiple tubing sections. V# indicates a valve. BS# indicates a bubble sensor or ultrasonic air detector. N# indicates a needle (e.g., a needle that injects sample into a sample holder). PS# indicates a pressure sensor (e.g., a reusable pressure sensor). Pump# indicates a fluid pump (e.g., a syringe pump with a disposable body and reusable drive). “Hb 12” indicates a sensor for hemoglobin (e.g., a dilution sensor that can detect hemoglobin optically).

The function of the valves, pumps, actuators, drivers, motors (e.g., the centrifuge motor), etc. described below is controlled by one or more controllers (e.g., the fluid system controller 405, the optical system controller 413, etc.) The controllers can include software, computer memory, electrical and mechanical connections to the controlled components, etc.

At the start of a measurement cycle, most lines, including a patient tube 512 (T1), an Hb sensor tube 528 (T4), an anticoagulant valve tube 534 (T3), and a sample cell 548 can be filled with saline that can be introduced into the system through the infusion tube 514 and the saline tube 516, and which can come from an infusion pump 518 and/or a saline bag 520. The infusion pump 518 and the saline bag 520 can be provided separately from the system 510. For example, a hospital can use existing saline bags and infusion pumps to interface with the described system. The infusion valve 521 can be open to allow saline to flow into the tube 512 (T1).

Before drawing a sample, the saline in part of the system 510 can be replaced with air. Thus, for example, the following valves can be closed: air valve 503 (PV0), the terg tank valve 559 (V7b), 566 (V3b), 523 (V0), 529 (V7a), and 563 (V2b). At the same time, the following valves can be open: valves 531 (V1a), 533 (V3a) and 577 (V4a). Simultaneously, a second pump 532 (pump #0) pumps air through system 510, pushing saline through tube 534 (T3) and sample cell 548 into a waste bladder 554.

Next, a sample can be drawn. With the valves 542 (PV1), 559 (V7b), and 561 (V4b) closed, a first pump 522 (pump #1) is actuated to draw sample fluid to be analyzed (e.g. blood) from a fluid source (e.g., a laboratory sample container, a living patient, etc.) up into the patient tube 512 (T1), through the tube past the two flanking portions of the open pinch-valve 523 (V0), through the first connector 524 (C1), into the looped tube 530, past the hemoglobin sensor 526 (Hb12), and into the Hb sensor tube 528 (T4). During this process, the valve 529 (V7a) and 523 (V0) are open to fluid flow, and the valves 531 (V1a), 533 (V3a), *42 (PV1), *59 (V7b), and 561 (V4b) can be closed and therefore block (or substantially block) fluid flow by pinching the tube.

Before drawing the sample, the tubes 512 (T1) and 528 (T4) are filled with saline and the hemoglobin (Hb) level is zero. The tubes that are filled with saline are in fluid communication with the sample source (e.g., the fluid source 402). The sample source can be the vessels of a living human or a pool of liquid in a laboratory sample container, for example. When the saline is drawn toward the first pump 522, fluid to be analyzed is also drawn into the system because of the suction forces in the closed fluid system. Thus, the first pump 522 draws a relatively continuous column of fluid that first comprises generally nondiluted saline, then a mixture of saline and sample fluid (e.g., blood), and then eventually nondiluted sample fluid. In the example illustrated here, the sample fluid is blood.

The hemoglobin sensor 526 (Hb12) detects the level of Hemoglobin in the sample fluid. As blood starts to arrive at the hemoglobin sensor 526 (Hb12), the hemoglobin level rises. A hemoglobin level can be selected, and the system can be pre-set to determine when that level is reached. A controller such as the fluid system controller 405 of FIG. 4 can be used to set and react to the pre-set value, for example. In some embodiments, when the sensed hemoglobin level reaches the pre-set value, substantially undiluted sample is present at the first connector 524 (C1). The preset value can depend, in part, on the length and diameter of any tubes and/or passages traversed by the sample. In some embodiments, the pre-set value can be reached after approximately 2 mL of fluid (e.g., blood) has been drawn from a fluid source. A nondiluted sample can be, for example, a blood sample that is not diluted with saline solution, but instead has the characteristics of the rest of the blood flowing through a patient's body. A loop of tubing 530 (e.g., a 1-mL loop) can be advantageously positioned as illustrated to help insure that undiluted fluid (e.g., undiluted blood) is present at the first connector 524 (C1) when the hemoglobin sensor 526 registers that the preset Hb threshold is crossed. The loop of tubing 530 provides additional length to the Hb sensor tube 528 (T4) to make it less likely that the portion of the fluid column in the tubing at the first connector 524 (C1) has advanced all the way past the mixture of saline and sample fluid, and the nondiluted blood portion of that fluid has reached the first connector 524 (C1).

In some embodiments, when nondiluted blood is present at the first connector 524 (C1), a sample is mixed with an anticoagulant and is directed toward the sample cell 548. An amount of anticoagulant (e.g., heparin) can be introduced into the tube 534 (T3), and then the undiluted blood is mixed with the anticoagulant. A heparin vial 538 (e.g., an insertable vial provided independently by the user of the system 510) can be connected to a tube 540. An anticoagulant valve 541 (which can be a shuttle valve, for example) can be configured to connect to both the tube 540 and the anticoagulant valve tube 534 (T3). The valve can open the tube 540 to a suction force (e.g., created by the pump 532), allowing heparin to be drawn from the vial 538 into the valve 541. Then, the anticoagulant valve 541 can slide the heparin over into fluid communication with the anticoagulant valve tube 534 (T3). The anticoagulant valve 541 can then return to its previous position. Thus, heparin can be shuttled from the tube 540 into the anticoagulant valve tube 534 (T3) to provide a controlled amount of heparin into the tube 534 (T3).

With the valves 542 (PV1), 559 (V7b), 561 (V4b), 523 (V0), 531 (V1a), 566 (V3b), and 563 (V2b) closed, and the valves 529 (V7a) and 553 (V3a) open, first pump 522 (pump #1) pushes the sample from tube 528 (T4) into tube 534 (T3), where the sample mixes with the heparin injected by the anticoagulant valve 541 as it flows through the system 510. The sample continues to flow until a bubble sensor 535 (B S9) indicates the presences of the bubble. In some embodiments, the volume of tube 534 (T3) from connector 524 (C1) to bubble sensor 535 (BS9) is a known amount, and may be, for example, approximately 100 microliters.

When bubble sensor 535 (BS9) indicates the presence of a sample, the remainder of the sampled blood can be returned to its source (e.g., the patient veins or arteries). The first pump 522 (pump #1) pushes the blood out of the Hb sensor tube 528 (T4) and back to the patient by opening the valve 523 (V0), closing the valves 531 (V1a) and 533 (V3a), and keeping the valve 529 (V7a) open. The Hb sensor tube 528 (T4) is preferably flushed with approximately 2 mL of saline. This can be accomplished by closing the valve 529 (V7a), opening the valve 542 (PV1), drawing saline from the saline source 520 into the tube 544, closing the valve 542 (PV1), opening the valve 529 (V7a), and forcing the saline down the Hb sensor tube 528 (T4) with the pump 522. In some embodiments, less than two minutes elapse between the time that blood is drawn from the patient and the time that the blood is returned to the patient.

Following return of the unused patient blood sample, the sample is pushed up the anticoagulant valve tube 534 (T3), through the second connector 546 (C2), and into the sample cell 548, which can be located on the centrifuge rotor 550. This fluid movement is facilitated by the coordinated action (either pushing or drawing fluid) of the pump E22 (pump #1), the pump E32 (pump #0), and the various illustrated valves. Pump movement and valve position corresponding to each stage of fluid movement can be coordinated by one ore multiple controllers, such as the fluid system controller 405 of FIG. 4.

After the unused sample is returned to the patient, the sample can be divided into separate slugs before being delivered into the sample cell 548. Thus, for example, valves 553 (V3a) and 531 (V1a) are opened, valves 523 (V0) and 529 (V7a) are closed, and the first pump 522 (pump #1) uses saline to push the sample towards sample cell 548. In some embodiments, the sample (for example 100 microliters) is divided into four “slugs” of sample, each separated by a small amount of air. As used herein, the term “slug” refers to a continuous column of fluid that can be relatively short. Slugs can be separated from one another by small amounts of air (or bubbles) that can be present at intervals in the tube. In some embodiments, the slugs are formed by injecting or drawing air into fluid in the first connector 546 (C2).

In some embodiments, when the leading edge of the sample reaches blood sensor 553 (BS14), a small amount of air (the first “bubble”) is injected at a connector 546 (C2), defining the first slug, which extends from the bubble sensor to the first bubble. In some embodiments, the valves 503 (PV0) and 559 (V7b) are closed, the valve 556 (V3b) is open, the pump 532 is actuated briefly to inject a first air bubble into the sample, and then valve 556 (V3b) is closed.

In some embodiments, the volume of the tube 534 (T3) from the connector 546 (C2) to the bubble sensor 552 (BS14) is less than the volume of tube 534 (T3) from the connector 524 (C1) to the bubble sensor 535 (BS9). Thus, for example and without limitation, the volume of the tube 534 (T3) from the connector 524 (C1) to the bubble sensor 535 (BS9) is approximately 100 μL, and the volume of the tube 534 (T3) from the connector 546 (C2) to the bubble sensor 552 (BS14) is approximately 15 μL. In some embodiments, four blood slugs are created. The first three blood slugs can have a volume of approximately 15 μL and the fourth can have a volume of approximately 35 μL.

A second slug can be prepared by opening the valves 553 (V3a) and 531 (V1a), closing the valves 523 (V0) and 529 (V7a), and operating the first pump 522 (pump #1) to push the first slug through a first sample cell holder interface tube 582 (N1), through the sample cell 548, through a second sample cell holder interface tube 584 (N2), and toward the waste bladder 554. When the first bubble reaches the bubble sensor 552 (BS 14), the first pump 522 (pump #1) is stopped, and a second bubble is injected into the sample, as before. A third slug can be prepared in the same manner as the second (pushing the second bubble to bubble sensor 552 (BS 14) and injecting a third bubble). After the injection of the third air bubble, the sample can be pushed through system 510 until the end of the sample is detected by bubble sensor 552 (BS 14). The system can be designed such that when the end of the sample reaches this point, the last portion of the sample (a fourth slug) is within the sample cell 548, and the pump 522 can stop forcing the fluid column through the anticoagulant valve tube 534 (T3) so that the fourth slug remains within the sample cell 548. Thus, the first three blood slugs can serve to flush any residual saline out the sample cell 548. The three leading slugs can be deposited in the waste bladder 554 by passing through the tube F56 (T6) and past the tube-flanking portions of the open pinch valve 557 (V4a).

In some embodiments, the fourth blood slug is centrifuged for two minutes at 7200 RPM. Thus, for example, the sample cell holder interface tubes 582 (N1) and 584 (N2) disconnect the sample cell 548 from the tubes 534 (T3) and 562 (T7), permitting the centrifuge rotor 550 and the sample cell 548 to spin together. Spinning separates a sample (e.g., blood) into its components, isolates the plasma, and positions the plasma in the sample cell 548 for measurement. The centrifuge 550 can be stopped with the sample cell 548 in a beam of radiation (not shown) for analysis. The radiation, a detector, and logic can be used to analyze the a portion of the sample (e.g., the plasma) spectroscopically (e.g., for glucose, lactate, or other analyte concentration).

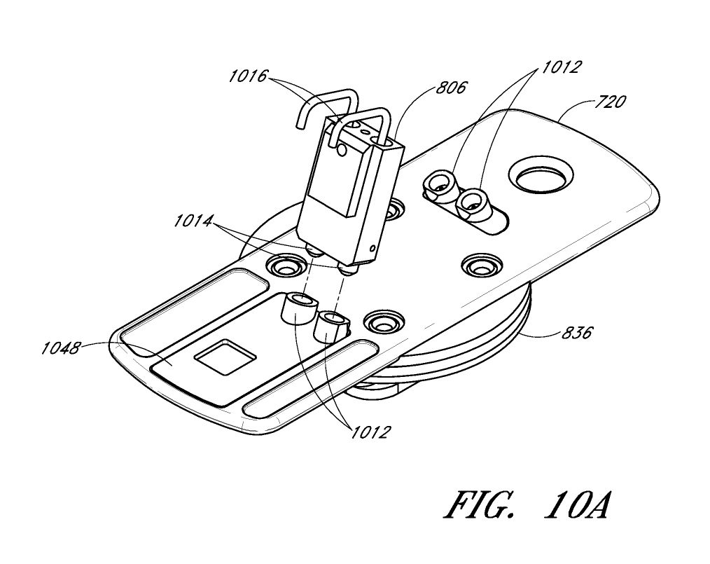

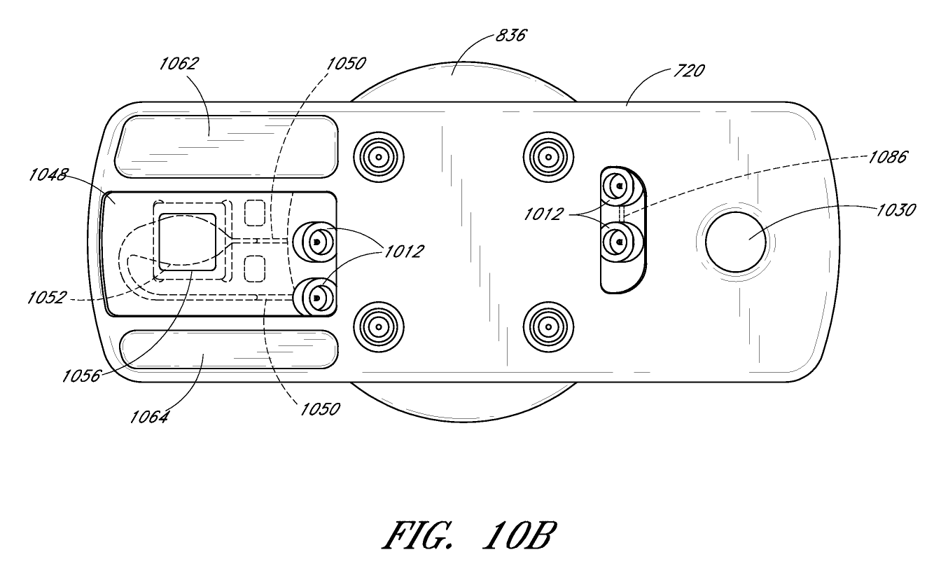

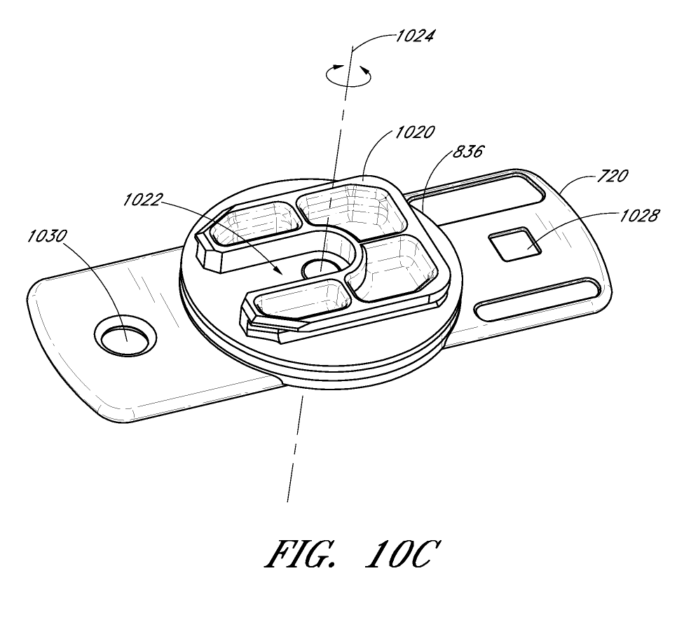

In some embodiments, portions of the system 510 that contain blood after the sample cell 548 has been provided with a sample are cleaned to prevent blood from clotting. Accordingly, the centrifuge rotor 550 can include two passageways for fluid that may be connected to the sample cell holder interface tubes 582 (N1) and 584 (N2). One passageway is sample cell 548, and a second passageway is a shunt 586. An embodiment of the shunt 586 is illustrated in more detail in FIG. 10B.

The shunt 586 can allow cleaner (e.g., tergazyme A) to flow through and clean the sample cell holder interface tubes without flowing through the sample cell 548. After the sample cell 548 is provided with a sample, the interface tubes 582 (N1) and 584 (N2) are disconnected from the sample cell 548, the centrifuge rotor 550 is rotated to align the shunt 586 with the interface tubes 582 (N1) and 584 (N2), and the interface tubes are connected with the shunt. With the shunt in place, the terg tank 559 is pressurized by the second pump 532 (pump #0) with valves 561 (V4b) and 563 (V2b) open and valves 557 (V4a) and 533 (V3a) closed to flush the cleaning solution back through the interface tubes 582 (N1) and 584 (N2) and into the waste bladder 554. Subsequently, saline can be drawn from the saline bag 520 for a saline flush. This flush pushes saline through the Hb sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (V1a), 566 (V3b), 563 (V2b), and 561 (V4b).

Following analysis, the second pump 532 (pump #0) flushes the sample cell 548 and sends the flushed contents to the waste bladder 554. This flush can be done with a cleaning solution from the terg tank 558. In some embodiments, the second pump 532 is in fluid communication with the terg tank tube 560 (T9) and the terg tank 558 because the terg tank valve 559 (V7b) is open. The second pump 532 forces cleaning solution from the terg tank 558 between the tube-flanking portions of the open pinch valve 561 and through the tube 562 (T7) when the valve 559 is open. The cleaning flush can pass through the sample cell 548, through the second connector 546, through the tube 564 (T5) and the open valve 563 (V2b), and into the waste bladder 554.

Subsequently, the first pump 522 (pump #1) can flush the cleaning solution out of the sample cell 548 using saline in drawn from the saline bag 520. This flush pushes saline through the Hb sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (V1a), 566 (V3b), 563 (V2b), and 561 (V4b).

When the fluid source is a living entity such as a patient, a low flow of saline (e.g., 1-5 mL/hr) is preferably moved through the patient tube 512 (T1) and into the patient to keep the patient's vessel open (e.g., to establish a keep vessel open, or “KVO” flow). This KVO flow can be temporarily interrupted when fluid is drawn into the fluid system 510. The source of this KVO flow can be the infusion pump 518, the third pump 568 (pump #3), or the first pump 522 (pump #1). In some embodiments, the infusion pump 518 can run continuously throughout the measurement cycle described above. This continuous flow can advantageously avoid any alarms that may be triggered if the infusion pump 518 senses that the flow has stopped or changed in some other way. In some embodiments, when the infusion valve 521 closes to allow pump 522 (pump #1) to withdraw fluid from a fluid source (e.g., a patient), the third pump 568 (pump #3) can withdraw fluid through the connector 570, thus allowing the infusion pump 518 to continue pumping normally as if the fluid path was not blocked by the infusion valve 521. If the measurement cycle is about two minutes long, this withdrawal by the third pump 568 can continue for approximately two minutes. Once the infusion valve 521 is open again, the third pump 568 (pump #3) can reverse and insert the saline back into the system at a low flow rate. Preferably, the time between measurement cycles is longer than the measurement cycle itself (e.g., longer than two minutes). Accordingly, the third pump 568 can insert fluid back into the system at a lower rate than it withdrew that fluid. This can help prevent an alarm by the infusion pump.

FIG. 5B schematically illustrates another embodiment of a fluid system that can be part of a system for withdrawing and analyzing fluid samples. In this embodiment, the anticoagulant valve 541 has been replaced with a syringe-style pump 588 (Pump Heparin) and a series of pinch valves around a junction between tubes. For example, a heparin pinch valve 589 (Vhep) can be closed to prevent flow from or to the pump 588, and a heparin waste pinch valve 590 can be closed to prevent flow from or to the waste container from this junction through the heparin waste tube 591. This embodiment also illustrates the shunt 592 schematically. Other differences from FIG. 5A include the check valve 593 located near the terg tank 558 and the patient loop 594. The reference letters D, for example, the one indicated at 595, refer to components that are advantageously located on the door. The reference letters M, for example, the one indicated at 596, refer to components that are advantageously located on the monitor. The reference letters B, for example, the one indicated at 597, refer to components that can be advantageously located on both the door and the monitor.

In some embodiments, the system 400 (see FIG. 4), the apparatus 100 (see FIG. 1), or even the monitoring device 102 (see FIG. 1) itself can also actively function not only to monitor analyte levels (e.g., glucose), but also to change analyte levels. Thus, the monitoring device 102 can be both a monitoring and an infusing device. For example, analyte levels in a patient can be adjusted directly (e.g., by infusing or extracting glucose) or indirectly (e.g., by infusing or extracting insulin). FIG. 5B illustrates one way of providing this function. The infusion pinch valve 598 (V8) can allow the port sharing pump 599 (compare to the third pump 568 (pump #3) in FIG. 5A) to serve two roles. In the first role, it can serve as a “port sharing” pump. The port sharing function is described with respect to the third pump 568 (pump #3) of FIG. 5A, where the third pump 568 (pump #3) can withdraw fluid through the connector 570, thus allowing the infusion pump 518 to continue pumping normally as if the fluid path was not blocked by the infusion valve 521. In the second role, the port sharing pump 599 can serve as an infusion pump. The infusion pump role allows the port sharing pump 599 to draw a substance (e.g., glucose, saline, etc.) from another source when the infusion pinch valve 598 is open, and then to infuse that substance into the system or the patient when the infusion pinch valve 598 is closed. This can occur, for example, in order to change the level of a substance in a patient in response to a reading by the monitor that the substance is too low. Other embodiments, such as those detailed in U.S. patent application Ser. Nos. 11/316,407 (OPTIS.154A), 11/316,212 (OPTIS.155A), and 11/316,684 (OPTIS.157A), can accomplish a similar function. These three patent applications are hereby incorporated by reference herein for all that they contain, and each is hereby made part of this specification. These three applications describe, intra alias an analytical device with a reversible infusion pump. The reversible infusion pump can interrupt the flow of the infusion fluid and draw a sample of blood for analysis.

Mechanical/Fluid System Interface

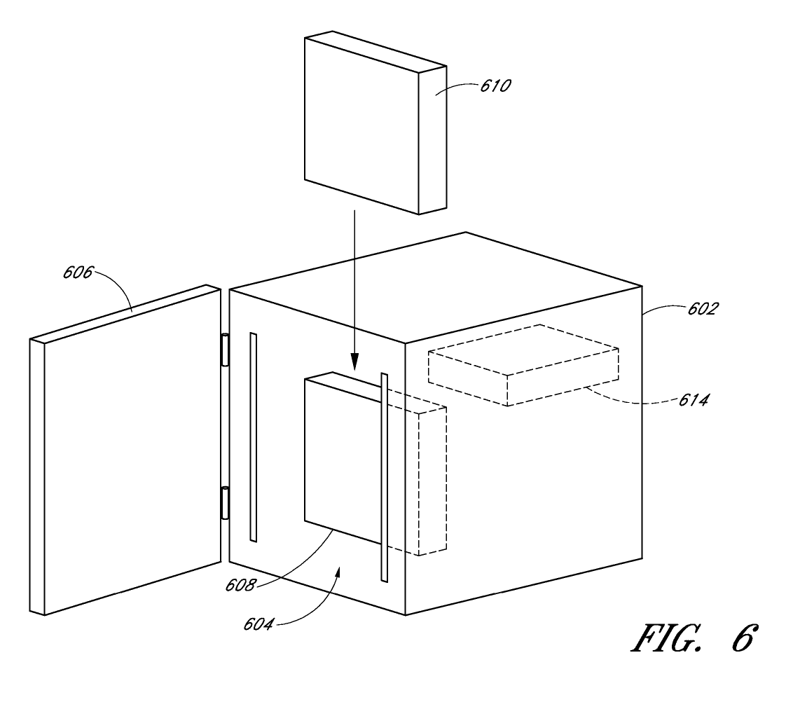

FIG. 6 is an oblique schematic depiction of a modular monitoring device 600, which can correspond to the monitoring device 102. The modular monitoring device 600 includes a body portion 602 having a receptacle 604, which can be accessed by moving a movable portion 606. The receptacle 604 can include connectors (e.g., rails, slots, protrusions, resting surfaces, etc.) with which a removable portion 610 can interface. In some embodiments, portions of a fluidic system that directly contact fluid are incorporated into one or more removable portions (e.g., one or more disposable cassettes, sample holders, tubing cards, etc.). For example, a removable portion 610 can house at least a portion of the fluid system 510 described previously, including portions that contact sample fluids, saline, detergent solution, and/or anticoagulant.

In some embodiments, a non-disposable fluid-handling subsystem 608 is disposed within the body portion 602 of the monitoring device 600. The first removable portion 610 can include one or more openings that allow portions of the non-disposable fluid-handling subsystem 608 to interface with the removable portion 610. For example, the non-disposable fluid-handling subsystem 608 can include one or more pinch valves that are designed to extend through such openings to engage one or more sections of tubing. When the first removable portion 610 is present in a corresponding first receptacle 604, actuation of the pinch valves can selectively close sections of tubing within the removable portion. The non-disposable fluid-handling subsystem 608 can also include one or more sensors that interface with connectors, tubing sections, or pumps located within the first removable portion 610. The non-disposable fluid-handling subsystem 608 can also include one or more actuators (e.g., motors) that can actuate moveable portions (e.g., the plunger of a syringe) that may be located in the removable portion F10. A portion of the non-disposable fluid-handling subsystem 608 can be located on or in the moveable portion F06 (which can be a door having a slide or a hinge, a detachable face portion, etc.).

In the embodiment shown in FIG. 6, the monitoring device 600 includes an optical system 614 disposed within the body portion 602. The optical system 614 can include a light source and a detector that are adapted to perform measurements on fluids within a sample holder (not shown). In some embodiments, the sample holder comprises a removable portion, which can be associated with or disassociated from the removable portion F10. The sample holder can include an optical window through which the optical system 614 can emit radiation for measuring properties of a fluid in the sample holder. The optical system 614 can include other components such as, for example, a power supply, a centrifuge motor, a filter wheel, and/or a beam splitter.

In some embodiments, the removable portion 610 and the sample holder are adapted to be in fluid communication with each other. For example, the removable portion 610 can include a retractable injector that injects fluids into a sample holder. In some embodiments, the sample holder can comprise or be disposed in a second removable portion (not shown). In some embodiments, the injector can be retracted to allow the centrifuge to rotate the sample holder freely.

The body portion 602 of the monitoring device 600 can also include one or more connectors for an external battery (not shown). The external battery can serve as a backup emergency power source in the event that a primary emergency power source such as, for example, an internal battery (not shown) is exhausted.

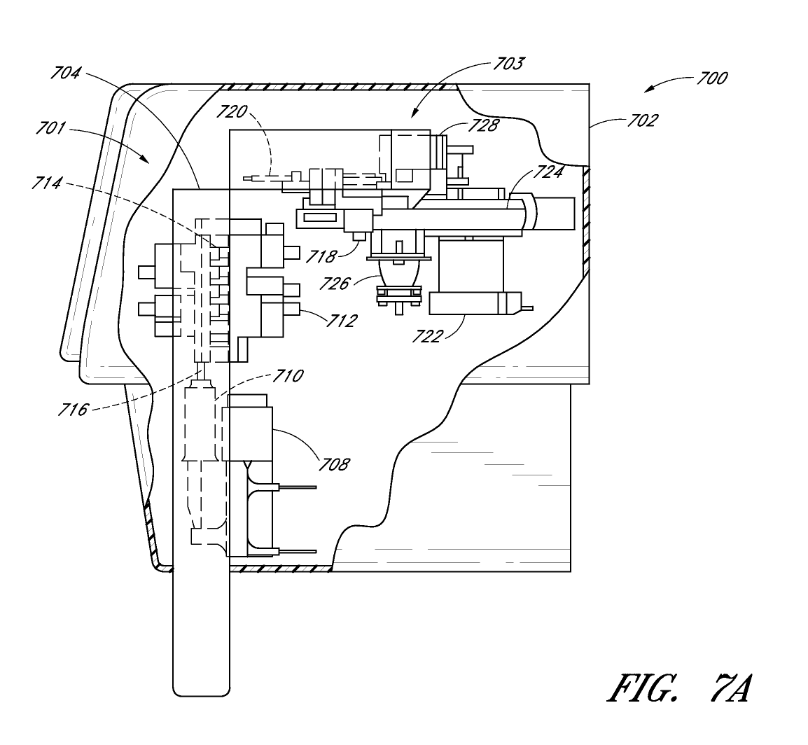

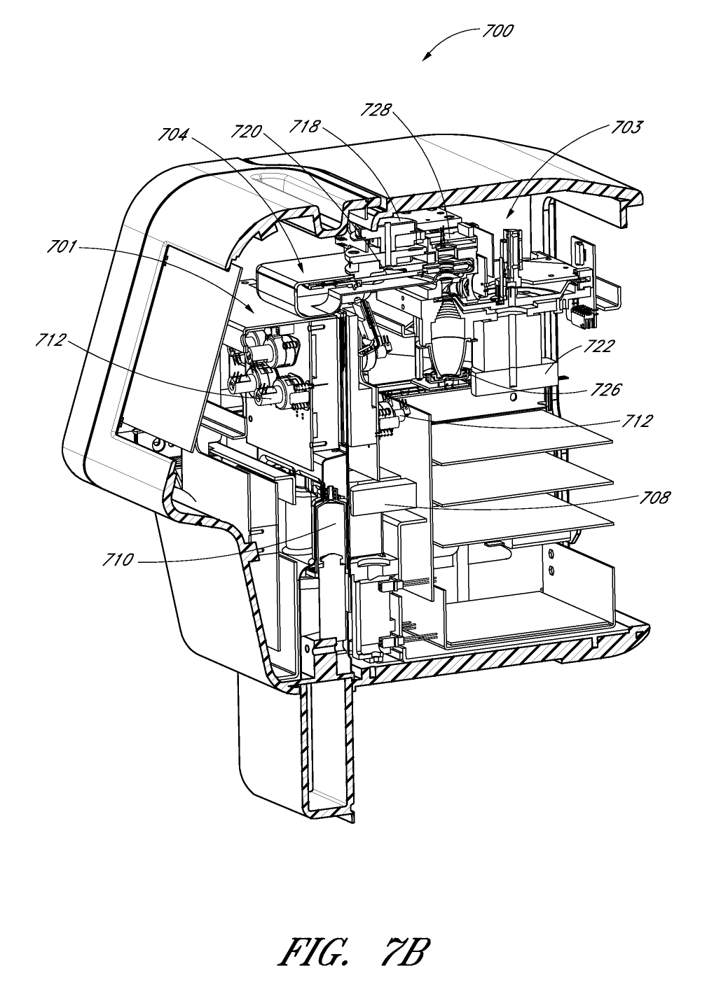

FIG. 6 shows an embodiment of a system having subcomponents illustrated schematically. By way of a more detailed (but nevertheless non-limiting) example, FIG. 7A and FIG. 7B show more details of the shape and physical configuration of a sample embodiment.

FIG. 7A shows a cut-away side view of a monitoring device 700 (which can correspond, for example, to the device 102 shown in FIG. 1). The device 700 includes a casing 702. The monitoring device 700 can have a fluid system. For example, the fluid system can have subsystems, and a portion or portions thereof can be disposable, as schematically depicted in FIG. 4. As depicted in FIG. 7A, the fluid system is generally located at the left-hand portion of the casing 702, as indicated by the reference 701. The monitoring device 700 can also have an optical system. In the illustrated embodiment, the optical system is generally located in the upper portion of the casing 702, as indicated by the reference 703. Advantageously, however, the fluid system 701 and the optical system 703 can both be integrated together such that fluid flows generally through a portion of the optical system 703, and such that radiation flows generally through a portion of the fluid system 701.

Depicted in FIG. 7A are examples of ways in which components of the device 700 mounted within the casing 702 can interface with components of the device 700 that comprise disposable portions. Not all components of the device 700 are shown in FIG. 7A. A disposable portion 704 having a variety of components is shown in the casing 702. In some embodiments, one or more actuators 708 housed within the casing 702, operate syringe bodies 710 located within a disposable portion 704. The syringe bodies 710 are connected to sections of tubing 716 that move fluid among various components of the system. The movement of fluid is at least partially controlled by the action of one or more pinch valves 712 positioned within the casing 702. The pinch valves 712 have arms 714 that extend within the disposable portion 704. Movement of the arms 714 can constrict a section of tubing 716.

In some embodiments, a sample cell holder 720 can engage a centrifuge motor 718 mounted within the casing 702 of the device 700. A filter wheel motor 722 disposed within the housing 702 rotates a filter wheel 724, and in some embodiments, aligns one or more filters with an optical path. An optical path can originate at a source 726 within the housing 702 that can be configured to emit a beam of radiation (e.g., infrared radiation, visible radiation, ultraviolet radiation, etc.) through the filter and the sample cell holder 720 and to a detector 728. A detector 728 can measure the optical density of the light when it reaches the detector.

FIG. 7B shows a cut-away perspective view of an alternative embodiment of a monitoring device 700. Many features similar to those illustrated in FIG. 7A are depicted in this illustration of an alternative embodiment. A fluid system 701 can be partially seen. The disposable portion 704 is shown in an operative position within the device. One of the actuators 708 can be seen next to a syringe body 710 that is located within the disposable portion 704. Some pinch valves 712 are shown next to a fluid-handling portion of the disposable portion 704. In this figure, an optical system 703 can also be partially seen. The sample holder 720 is located underneath the centrifuge motor 718. The filter wheel motor 722 is positioned near the radiation source 726, and the detector 728 is also illustrated.

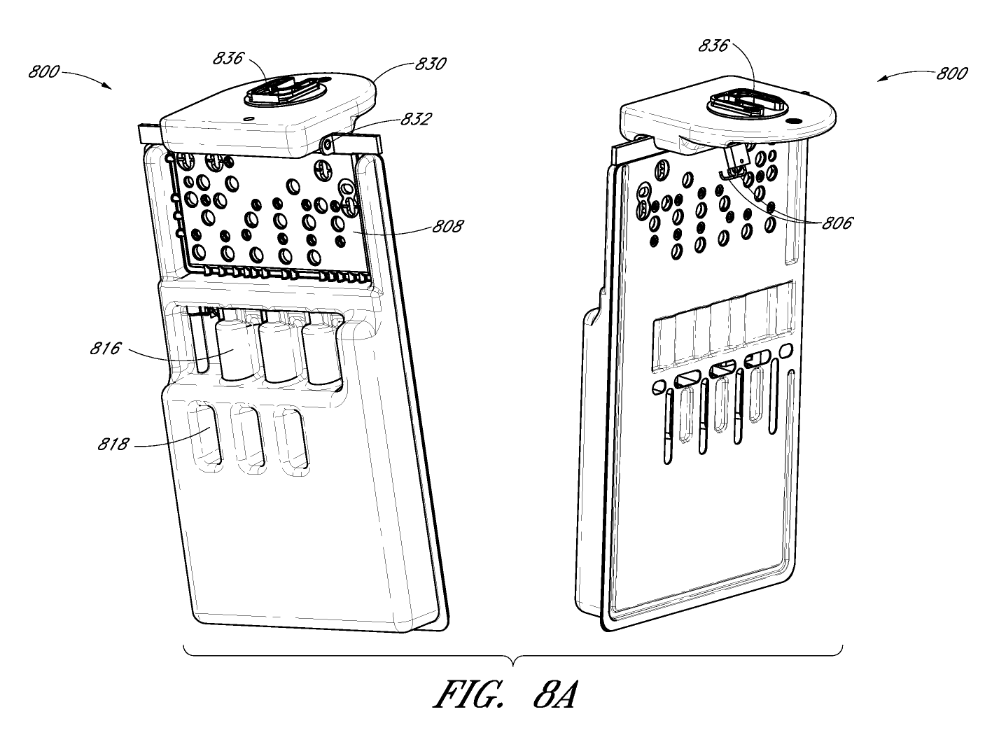

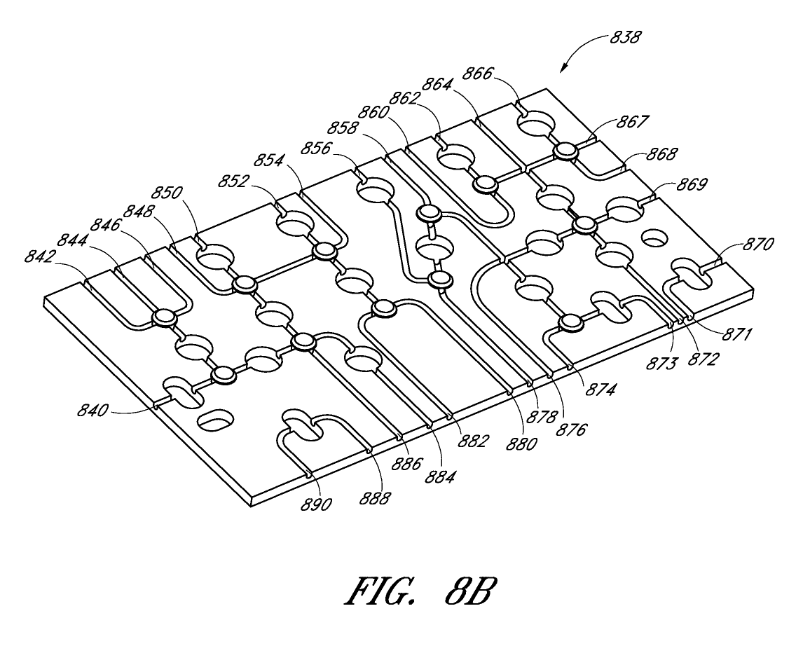

FIG. 8A illustrates two views of a disposable cartridge 800 that can interface with a fluid system such as the fluid system 510 of FIG. 5A. The disposable cartridge 800 can be configured for insertion into a receptacle of the device 700 of FIG. 7A and/or the device 700 shown in FIG. 7B. The disposable cartridge 800 can fill the role of the removable portion 610 of FIG. 6, for example. In some embodiments, the disposable cartridge 800 can be used for a system having only one disposable subsystem, making it a simple matter for a health care provider to replace and/or track usage time of the disposable portion. In some embodiments, the cartridge 800 includes one or more features that facilitate insertion of the cartridge 800 into a corresponding receptacle. For example, the cartridge 800 can be shaped so as to promote insertion of the cartridge 800 in the correct orientation. The cartridge 800 can also include labeling or coloring affixed to or integrated with the cartridge's exterior casing that help a handler insert the cartridge 800 into a receptacle properly.

The cartridge 800 can include one or more ports for connecting to material sources or receptacles. Such ports can be provided to connect to, for example, a saline source, an infusion pump, a sample source, and/or a source of gas (e.g., air, nitrogen, etc.). The ports can be connected to sections of tubing within the cartridge 800. In some embodiments, the sections of tubing are opaque or covered so that fluids within the tubing cannot be seen, and in some embodiments, sections of tubing are transparent to allow interior contents (e.g., fluid) to be seen from outside.

The cartridge 800 shown in FIG. 8A can include a sample injector 806. The sample injector 806 can be configured to inject at least a portion of a sample into a sample holder (see, e.g., the sample cell 548), which can also be incorporated into the cartridge 800. The sample injector 806 may include, for example, the sample cell holder interface tubes 582 (N1) and 584 (N2) of FIG. 5A, embodiments of which are also illustrated in FIG. 10A.

The housing of the cartridge 800 can include a tubing portion 808 containing within it a card having one or more sections of tubing. In some embodiments, the body of the cartridge 800 includes one or more apertures 809 through which various components, such as, for example, pinch valves and sensors, can interface with the fluid-handling portion contained in the cartridge 800. The sections of tubing found in the tubing portion 808 can be aligned with the apertures 809 in order to implement at least some of the functionality shown in the fluid system 510 of FIG. 5A.

The cartridge 800 can include a pouch space (not shown) that can comprise one or more components of the fluid system 510. For example, one or more pouches and/or bladders can be disposed in the pouch space (not shown). In some embodiments, a cleaner pouch and/or a waste bladder can be housed in a pouch space. The waste bladder can be placed under the cleaner pouch such that, as detergent is removed from the cleaner pouch, the waste bladder has more room to fill. The components placed in the pouch space (not shown) can also be placed side-by-side or in any other suitable configuration.

The cartridge 800 can include one or more pumps 816 that facilitate movement of fluid within the fluid system 510. Each of the pump housings 816 can contain, for example, a syringe pump having a plunger. The plunger can be configured to interface with an actuator outside the cartridge 800. For example, a portion of the pump that interfaces with an actuator can be exposed to the exterior of the cartridge 800 housing by one or more apertures 818 in the housing.

The cartridge 800 can have an optical interface portion 830 that is configured to interface with (or comprise a portion of) an optical system. In the illustrated embodiment, the optical interface portion 830 can pivot around a pivot structure 832. The optical interface portion 830 can house a sample holder (not shown) in a chamber that can allow the sample holder to rotate. The sample holder can be held by a centrifuge interface 836 that can be configured to engage a centrifuge motor (not shown). When the cartridge 800 is being inserted into a system, the orientation of the optical interface portion 830 can be different than when it is functioning within the system.

In some embodiments, the disposable cartridge 800 is designed for single patient use. The cartridge 800 may also be designed for replacement after a period of operation. For example, in some embodiments, if the cartridge 800 is installed in a continuously operating monitoring device that performs four measurements per hour, the waste bladder may become filled or the detergent in the cleaner pouch depleted after about three days. The cartridge 800 can be replaced before the detergent and waste bladder are exhausted.

The cartridge 800 can be configured for easy replacement. For example, in some embodiments, the cartridge 800 is designed to have an installation time of only several minutes. For example, the cartridge can be designed to be installed in less than about five minutes. During installation, various fluid lines contained in the cartridge 800 can be primed by automatically filling the fluid lines with saline. The saline can be mixed with detergent powder from the cleaner pouch in order to create a cleaning solution.

The cartridge 800 can also be designed to have a relatively brief shut down time. For example, the shut down process can be configured to take less than about five minutes. The shut down process can include flushing the patient line; sealing off the insulin pump connection, the saline source connection, and the sample source connection; and taking other steps to decrease the risk that fluids within the used cartridge 800 will leak after disconnection from the monitoring device.

Some embodiments of the cartridge 800 can comprise a flat package to facilitate packaging, shipping, sterilizing, etc. Advantageously, however, some embodiments can further comprise a hinge or other pivot structure. Thus, as illustrated, an optical interface portion 830 can be pivoted around a pivot structure *932 to generally align with the other portions of the cartridge 800. The cartridge can be provided to a medical provider sealed in a removable wrapper, for example.

In some embodiments, the cartridge 800 is designed to fit within standard waste containers found in a hospital, such as a standard biohazard container. For example, the cartridge 800 can be less than one foot long, less than one foot wide, and less than two inches thick. In some embodiments, the cartridge 800 is designed to withstand a substantial impact, such as that caused by hitting the ground after a four foot drop, without damage to the housing or internal components. In some embodiments, the cartridge 800 is designed to withstand significant clamping force applied to its casing. For example, the cartridge 800 can be built to withstand five pounds per square inch of force without damage. In some embodiments, the cartridge 800 is non pyrogenic and/or latex free.

FIG. 8B illustrates an embodiment of a fluid-routing card 838 that can be part of the removable cartridge of FIG. 8A. For example, the fluid-routing card 838 can be located generally within the tubing portion 808 of the cartridge 800. The fluid-routing card 838 can contain various passages and/or tubes through which fluid can flow as described with respect to FIG. 5A and/or FIG. 5B, for example. Thus, the illustrated tube opening openings can be in fluid communication with the following fluidic components, for example:

| Tube Opening | |
|---|---|
| Reference | |
| Numeral | Can Be In Fluid Communication With |
| 842 | third pump 568 (pump #3) |
| 844 | infusion pump 518 |
| 846 | presx |
| 848 | air pump |
| 850 | vent |
| 852 | detergent (e.g., tergazyme) source or waste tube |
| 854 | presx |
| 856 | detergent (e.g., tergazyme) source or waste tube |
| 858 | waste receptacle |
| 860 | first pump 522 (pump #1) (e.g., a saline pump) |
| 862 | saline source or waste tube |
| 864 | anticoagulant (e.g., heparin) pump (see FIG. 5B) |
| | and/or shuttle valve |
| 866 | detergent (e.g., tergazyme) source or waste tube |
| 867 | presx |
| 868 | Hb sensor tube 528 (T4) |
| 869 | tube 536 (T2) |
| 870 | Hb sensor tube 528 (T4) |
| 871 | Hb sensor tube 528 (T4) |
| 872 | anticoagulant (e.g., heparin) pump |
| 873 | T17 (see FIG. 5B) |
| 874 | Sample cell holder interface tube 582 (N1) |
| 876 | anticoagulant valve tube 534 (T3) |
| 878 | Sample cell holder interface tube 584 (N2) |
| 880 | T17 (see FIG. 5B) |
| 882 | anticoagulant valve tube 534 (T3) |
| 884 | Hb sensor tube 528 (T4) |
| 886 | tube 536 (T2) |
| 888 | anticoagulant valve tube 534 (T3) |
| 890 | anticoagulant valve tube 534 (T3) |

The depicted fluid-routing card 838 can have additional openings that allow operative portions of actuators and/or valves to protrude through the fluid-routing card 838 and interface with the tubes.

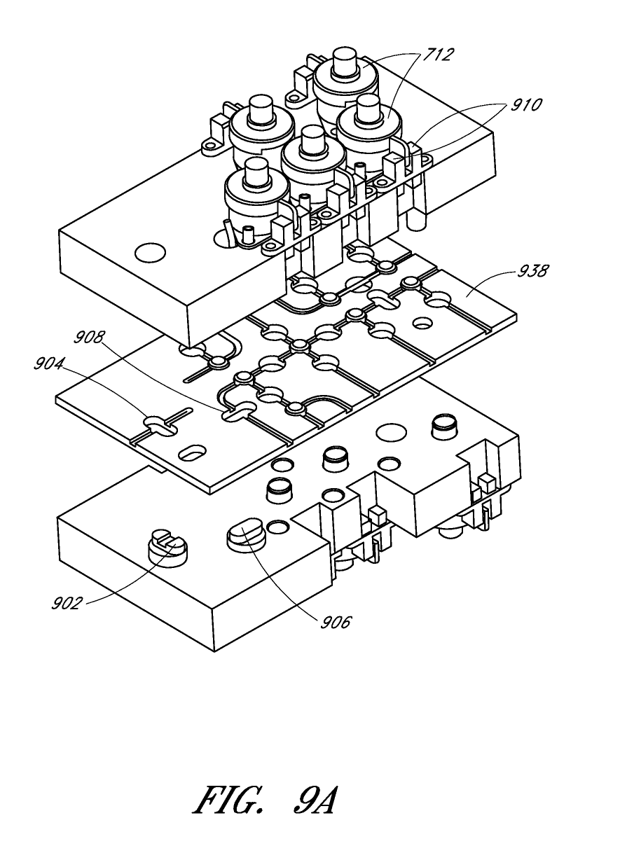

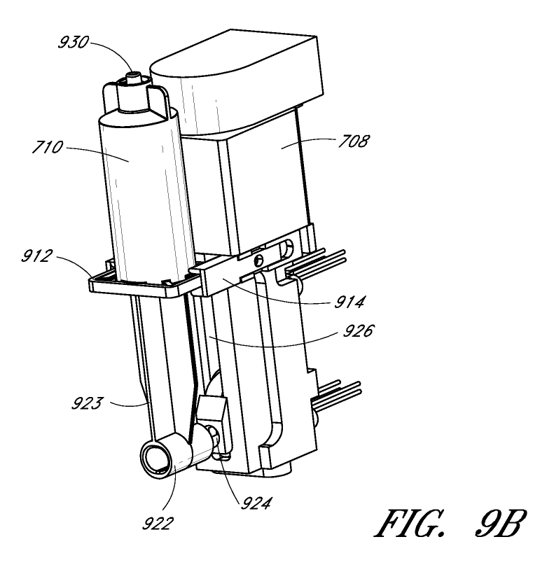

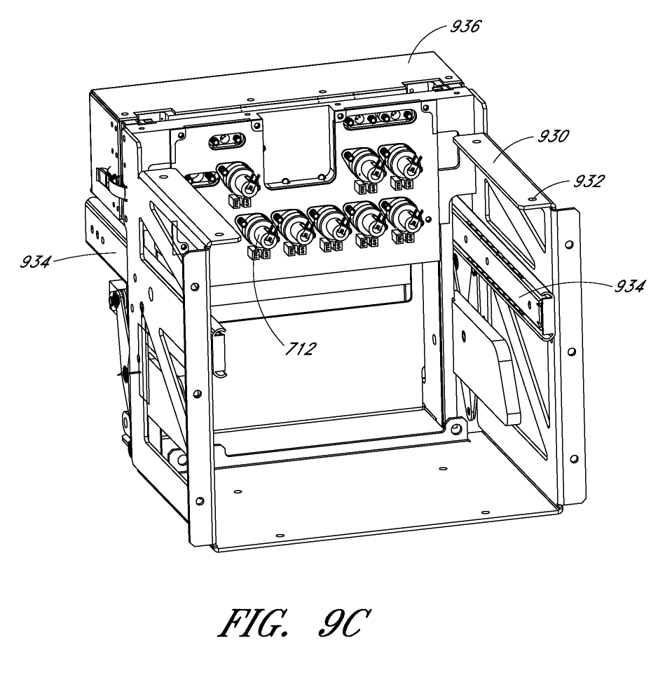

FIG. 9A illustrates how actuators, which can sandwich the fluid-routing card 838 between them, can interface with the fluid-routing card 838 of FIG. 8B. Pinch valves 712 can have an actuator portion that protrudes away from the fluid-routing card 838 containing a motor. Each motor can correspond to a pinch platen 902, which can be inserted into a pinch platen receiving hole 904. Similarly, sensors, such as a bubble sensor 906 can be inserted into receiving holes (e.g., the bubble sensor receiving hole 908). Movement of the pinch valves 712 can be detected by the position sensors 910.

FIG. 9B illustrates an actuator 708 that is connected to a corresponding syringe body 710. The actuator 708 is an example of one of the actuators 708 that is illustrated in FIG. 7A and in FIG. 7B, and the syringe body 710 is an example of one of the syringe bodies 710 that are visible in FIG. 7A and in FIG. 7B. A ledge portion 912 of the syringe body 710 can be engaged (e.g., slid into) a corresponding receiving portion 914 in the actuator 708. In some embodiments, the receiving portion 914 can slide outward to engage the stationary ledge portion 912 after the disposable cartridge 704 is in place. Similarly, a receiving tube 922 in the syringe plunger 923 can be slide onto (or can receive) a protruding portion 924 of the actuator 708. The protruding portion 924 can slide along a track 926 under the influence of a motor inside the actuator 708, thus actuating the syringe plunger 923 and causing fluid to flow into or out of the syringe tip 930.

FIG. 9C shows a rear perspective view of internal scaffolding 930 and the protruding bodies of some pinch valves 712. The internal scaffolding 930 can be formed from metal and can provide structural rigidity and support for other components. The scaffolding 930 can have holes 932 into which screws can be screwed or other connectors can be inserted. In some embodiments, a pair of sliding rails 934 can allow relative movement between portions of an analyzer. For example, a slidable portion 936 (which can correspond to the movable portion 606, for example) can be temporarily slid away from the scaffolding 930 of a main unit in order to allow an insertable portion (e.g., the cartridge 704) to be inserted.

FIG. 10A shows an underneath perspective view of the sample cell holder 720, which is attached to the centrifuge interface 836. The sample cell holder 720 can have an opposite side (see FIG. 10C) that allows it to slide into a receiving portion of the centrifuge interface 836. The sample cell holder 720 can also have receiving nubs 1012 that provide a pathway into a sample cell 1048 held by the sample cell holder 720. The receiving nubs 1012 can receive and or dock with fluid nipples 1014. The fluid nipples 1014 can protrude at an angle from the sample injector 806, which can in turn protrude from the cartridge 800 (see FIG. 8A). The tubes 1016 shown protruding from the other end of the sample injector 806 can be in fluid communication with the sample cell holder interface tubes 582 (N1) and 584 (N2) (see FIG. 5A and FIG. 5B), as well as 874 and 878 (see FIG. 8B).

FIG. 10B shows a plan view of the sample cell holder 720 with hidden and/or non-surface portions illustrated using dashed lines. The receiving nubs 1012 at the left communicate with passages 1050 inside the sample cell 1048 (which can correspond, for example to the sample cell 548 of FIG. 5A). The passages widen out into a wider portion 1052 that corresponds to a window 1056. The window 1056 and the wider portion 1052 can be configured to house the sample when radiation is emitted along a pathlength that is generally non-parallel to the sample cell 1048. The window 1056 can allow calibration of the instrument with the sample cell 1048 in place, even before a sample has arrived in the wider portion 1052.

An opposite opening 1030 can provide an alternative optical pathway between a radiation source and a radiation detector and may be used, for example, for obtaining a calibration measurement of the source and detector without an intervening window or sample. Thus, the opposite opening 1030 can be located generally at the same radial distance from the axis of rotation as the window 1056.

The receiving nubs 1012 at the right communicate with a shunt passage 1086 inside the sample cell holder 720 (which can correspond, for example to the shunt 586 of FIG. 5A).

Other features of the sample cell holder 720 can provide balancing properties for even rotation of the sample cell holder 720. For example, the wide trough 1062 and the narrower trough 1064 can be sized or otherwise configured so that the weight and/or mass of the sample cell holder 720 is evenly distributed from left to right in the view of FIG. JB, and/or from top to bottom in this view of FIG. JB.

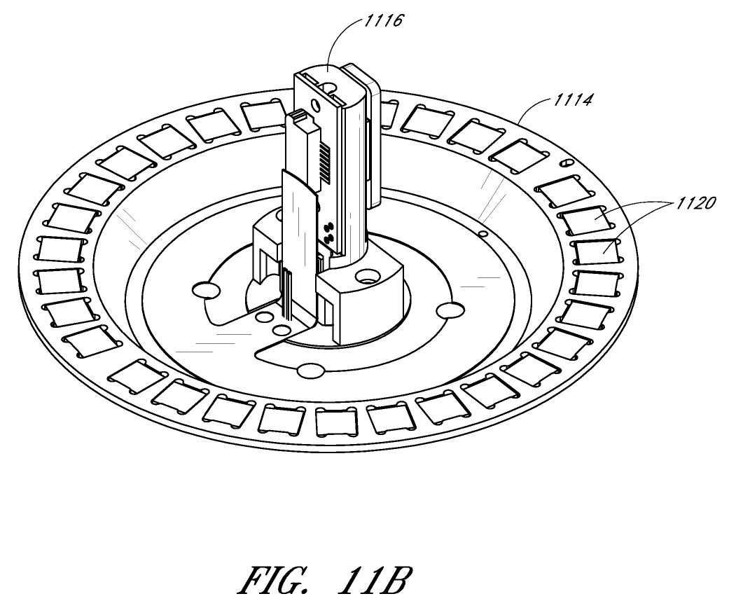

FIG. 10C shows a top perspective view of the centrifuge interface 836 connected to the sample cell holder 720. The centrifuge interface 836 can have a bulkhead 1020 with a rounded slot 1022 into which an actuating portion of a centrifuge can be slid from the side. The centrifuge interface 836 can thus be spun about an axis 1024, along with the sample cell holder 720, causing fluid (e.g., whole blood) within the sample cell 1048 to separate into concentric strata, according to relative density of the fluid components (e.g., plasma, red blood cells, buffy coat, etc.), within the sample cell 1048. The sample cell holder 720 can be transparent, or it can at least have transparent portions (e.g., the window 1056 and/or the opposite opening 1030) through which radiation can pass, and which can be aligned with an optical pathway between a radiation source and a radiation detector (see FIG. 12).

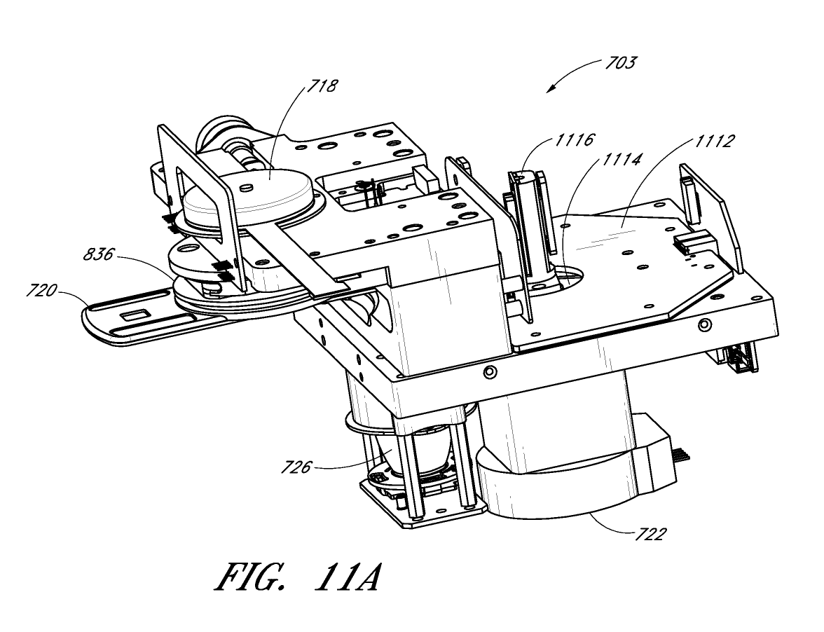

FIG. 11A shows a perspective view of an example optical system 703. Such a system can be integrated with other systems as shown in FIG. 7B, for example. The optical system 703 can fill the role of the optical system 412, and it can be integrated with and/or adjacent to a fluid system (e.g., the fluid-handling system 404 or the fluid system 701). The sample cell holder 720 can be seen attached to the centrifuge interface 836, which is in turn connected to, and rotatable by the centrifuge motor 718. A filter wheel housing 1112 is attached to the filter wheel motor 722 and encloses a filter wheel 1114. A protruding shaft assembly 1116 can be connected to the filter wheel 1114. The filter wheel 1114 can have multiple filters (see FIG. 11B). The radiation source 726 is aligned to transmit radiation through a filter in the filter wheel 1114 and then through a portion of the sample cell holder 720. Transmitted and/or reflected and/or scattered radiation can then be detected by a radiation detector.

FIG. 11B shows a view of the filter wheel 1114 when it is not located within the filter wheel housing 1112 of the optical system 703. Additional features of the protruding shaft assembly 1116 can be seen, along with multiple filters 1120. In some embodiments, the filters 1120 can be removably and/or replaceably inserted into the filter wheel 1114.

Spectroscopy

As described above with reference to FIG. 4, the system 400 comprises the optical system 412 for analysis of a fluid sample. In various embodiments, the optical system 412 comprises one or more optical components including, for example, a spectrometer, a photometer, a reflectometer, or any other suitable device for measuring optical properties of the fluid sample. The optical system 412 may perform one or more optical measurements on the fluid sample including, for example, measurements of transmittance, absorbance, reflectance, scattering, and/or polarization. The optical measurements may be performed in one or more wavelength ranges including, for example, infrared (IR) and/or optical wavelengths. As described with reference to FIG. 4 (and further described below), the measurements from the optical system 412 are communicated to the algorithm processor 416 for analysis. For example, In some embodiments the algorithm processor 416 computes concentration of analyte(s) (and/or interferent(s)) of interest in the fluid sample. Analytes of interest include, e.g., glucose and lactate in whole blood or blood plasma.

FIG. 12 schematically illustrates an embodiment of the optical system 412 that comprises a spectroscopic analyzer 1210 adapted to measure spectra of a fluid sample such as, for example, blood or blood plasma. The analyzer 1210 comprises an energy source 1212 disposed along an optical axis X of the analyzer 1210. When activated, the energy source 1212 generates an electromagnetic energy beam E, which advances from the energy source 1212 along the optical axis X. In some embodiments, the energy source 1212 comprises an infrared energy source, and the energy beam E comprises an infrared beam. In some embodiments, the infrared energy beam E comprises a mid-infrared energy beam or a near-infrared energy beam. In some embodiments, the energy beam E may include optical and/or radio frequency wavelengths.

The energy source 1212 may comprise a broad-band and/or a narrow-band source of electromagnetic energy. In some embodiments, the energy source 1212 comprises optical elements such as, e.g., filters, collimators, lenses, mirrors, etc., that are adapted to produce a desired energy beam E. For example, in some embodiments, the energy beam E is an infrared beam in a wavelength range between about 2 μm and 20 μm. In some embodiments, the energy beam E comprises an infrared beam in a wavelength range between about 4 μm and 10 μm. In the infrared wavelength range, water generally is the main contributor to the total absorption together with features from absorption of other blood components, particularly in the 6 μm-10 μm range. The 4 μm to 10 μm wavelength band has been found to be advantageous for determining glucose concentration, because glucose has a strong absorption peak structure from about 8.5 μm to 10 μm, whereas most other blood components have a relatively low and flat absorption spectrum in the 8.5 μm to 10 μm range. Two exceptions are water and hemoglobin, which are interferents in this range.

The energy beam E may be temporally modulated to provide increased signal-to-noise ratio (S/N) of the measurements provided by the analyzer 1210 as further described below. For example, in some embodiments, the beam E is modulated at a frequency of about 10 Hz or in a range from about 1 Hz to about 30 Hz. A suitable energy source 1212 may be an electrically modulated thin-film thermoresistive element such as the HawkEye IR-50 available from Hawkeye Technologies of Milford, Conn.

As depicted in FIG. 12, the energy beam E propagates along the optical axis X and passes through an aperture 1214 and a filter 1215 thereby providing a filtered energy beam E_{f}. The aperture 1214 helps collimate the energy beam E and may include one or more filters adapted to reduce the filtering burden of the filter 1215. For example, the aperture 1214 may comprise a broadband filter that substantially attenuates beam energy outside a wavelength band between about 4 μm to about 10 μm. The filter 1215 may comprise a narrow-band filter that substantially attenuates beam energy having wavelengths outside of a filter passband (which may be tunable or user-selectable in some embodiments). The filter passband may be specified by a half-power bandwidth (“HPBW”). In some embodiments, the filter 1215 may have an HPBW in a range from about 0.01 μm to about 1 μm. In some embodiments, the bandwidths are in a range from about 0.1 μm to 0.35 μm. Other filter bandwidths may be used. The filter 1215 may comprise a varying-passband filter, an electronically tunable filter, a liquid crystal filter, an interference filter, and/or a gradient filter. In some embodiments, the filter 1215 comprises one or a combination of a grating, a prism, a monochrometer, a Fabry-Perot etalon, and/or a polarizer. Other optical elements as known in the art may be utilized as well.

In the embodiment shown in FIG. 12, the analyzer 1210 comprises a filter wheel assembly 1221 configured to dispose one or more filters 1215 along the optical axis X. The filter wheel assembly 1221 comprises a filter wheel 1218, a filter wheel motor 1216, and a position sensor 1220. The filter wheel 1218 may be substantially circular and have one or more filters 1215 or other optical elements (e.g., apertures, gratings, polarizers, mirrors, etc.) disposed around the circumference of the wheel 1218. In some embodiments, the number of filters 1215 in the filter wheel 1216 may be, for example, 1, 2, 5, 10, 15, 20, 25, or more. The motor 1216 is configured to rotate the filter wheel 1218 to dispose a desired filter 1215 (or other optical element) in the energy beam E so as to produce the filtered beam E_{f}. In some embodiments, the motor 1216 comprises a stepper motor. The position sensor 1220 determines the angular position of the filter wheel 1216, and communicates a corresponding filter wheel position signal to the algorithm processor 416, thereby indicating which filter 1215 is in position on the optical axis X. In various embodiments, the position sensor 1220 may be a mechanical, optical, and/or magnetic encoder. An alternative to the filter wheel 1218 is a linear filter translated by a motor. The linear filter may include an array of separate filters or a single filter with properties that change along a linear dimension.

The filter wheel motor 1216 rotates the filter wheel 1218 to position the filters 1215 in the energy beam E to sequentially vary the wavelengths or the wavelength bands used to analyze the fluid sample. In some embodiments, each individual filter 1215 is disposed in the energy beam E for a dwell time during which optical properties in the passband of the filter are measured for the sample. The filter wheel motor 1216 then rotates the filter wheel 1218 to position another filter 1215 in the beam E. In some embodiments, 25 narrow-band filters are used in the filter wheel 1218, and the dwell time is about 2 seconds for each filter 1215. A set of optical measurements for all the filters can be taken in about 2 minutes, including sampling time and filter wheel movement. In some embodiments, the dwell time may be different for different filters 1215, for example, to provide a substantially similar S/N ratio for each filter measurement. Accordingly, the filter wheel assembly 1221 functions as a varying-passband filter that allows optical properties of the sample to be analyzed at a number of wavelengths or wavelength bands in a sequential manner.

In some embodiments of the analyzer 1210, the filter wheel 1218 includes 25 finite-bandwidth infrared filters having a Gaussian transmission profile and full-width half-maximum (FWHM) bandwidth of 28 cm⁻¹ corresponding to a bandwidth that varies from 0.14 μm at 7.08 μm to 0.28 μm at 10 μm. The central wavelength of the filters are, in microns: 7.082, 7.158, 7.241, 7.331, 7.424, 7.513, 7.605, 7.704, 7.800, 7.905, 8.019, 8.150, 8.271, 8.598, 8.718, 8.834, 8.969, 9.099, 9.217, 9.346, 9.461, 9.579, 9.718, 9.862, and 9.990.

With further reference to FIG. 12, the filtered energy beam E_{f} propagates to a beamsplitter 1222 disposed along the optical axis X. The beamsplitter 1222 separates the filtered energy beam E_{f} into a sample beam Eₛ and a reference beam Eᵣ. The reference beam Eᵣ propagates along a minor optical axis Y, which in this embodiment is substantially orthogonal to the optical axis X. The energies in the sample beam Eₛ and the reference beam Eᵣ may comprise any suitable fraction of the energy in the filtered beam E_{f}. For example, in some embodiments, the sample beam Eₛ comprises about 80%, and the reference beam Eᵣ comprises about 20%, of the filtered beam energy E_{f}. A reference detector 1236 is positioned along the minor optical axis Y. An optical element 1234, such as a lens, may be used to focus or collimate the reference beam Eᵣ onto the reference detector 1236. The reference detector 1236 provides a reference signal, which can be used to monitor fluctuations in the intensity of the energy beam E emitted by the source 1212. Such fluctuations may be due to drift effects, aging, wear, or other imperfections in the source 1212. The algorithm processor 416 may utilize the reference signal to identify changes in properties of the sample beam Eₛ that are attributable to changes in the emission from the source 1212 and not to the properties of the fluid sample. By so doing, the analyzer 1210 may advantageously reduce possible sources of error in the calculated properties of the fluid sample (e.g., concentration). In other embodiments of the analyzer 1210, the beamsplitter 1222 is not used, and substantially all of the filtered energy beam E_{f} propagates to the fluid sample.

As illustrated in FIG. 12, the sample beam Eₛ propagates along the optical axis X, and a relay lens 1224 transmits the sample beam Eₛ into a sample cell 1248 so that at least a fraction of the sample beam Eₛ is transmitted through at least a portion of the fluid sample in the sample cell 1248. A sample detector 1230 is positioned along the optical axis X to measure the sample beam Eₛ that has passed through the portion of the fluid sample. An optical element 1228, such as a lens, may be used to focus or collimate the sample beam Eₛ onto the sample detector 1230. The sample detector 1230 provides a sample signal that can be used by the algorithm processor 416 as part of the sample analysis.

In the embodiment of the analyzer 1210 shown in FIG. 12, the sample cell 1248 is located toward the outer circumference of the centrifuge wheel 1250 (which can correspond, for example, to the sample cell holder 720 described herein). The sample holder 1248 preferably comprises windows that are substantially transmissive to energy in the sample beam Eₛ. For example, in implementations using mid-infrared energy, the windows may comprise calcium fluoride. As described herein with reference to FIG. 5A, the sample holder 1248 is in fluid communication with an injector system that permits filling the sample holder 1248 with a fluid sample (e.g., whole blood) and flushing the sample holder 1248 (e.g., with saline or a detergent). The injector system may disconnect after filling the sample holder 1248 with the fluid sample to permit free spinning of the centrifuge wheel 1250.

The centrifuge wheel 1250 can be spun by a centrifuge motor 1226. In some embodiments of the analyzer 1210, the fluid sample (e.g., a whole blood sample) is spun at about 7200 rpm for about 2 minutes to separate blood plasma for spectral analysis. In some embodiments, an anti-clotting agent such as heparin may be added to the fluid sample before centrifuging to reduce clotting. With reference to FIG. 12, the centrifuge wheel 1250 is rotated to a position where the sample cell 1248 intercepts the sample beam Eₛ, allowing energy to pass through the sample cell *48 to the sample detector 1230.

The embodiment of the analyzer 1210 illustrated in FIG. 12 advantageously permits direct measurement of the concentration of analytes in the plasma sample rather than by inference of the concentration from measurements of a whole blood sample. An additional advantage is that relatively small volumes of fluid may be spectroscopically analyzed. For example, in some embodiments the fluid sample volume is between about 1 μL and 80 μL and is about 25 μL in some embodiments. In some embodiments, the sample holder 1248 is disposable and is intended for use with a single patient or for a single measurement.

In some embodiments, the reference detector 1236 and the sample detector 1230 comprise broadband pyroelectric detectors. As known in the art, some pyroelectric detectors are sensitive to vibrations. Thus, for example, the output of a pyroelectric infrared detector is the sum of the exposure to infrared radiation and to vibrations of the detector. The sensitivity to vibrations, also known as “microphonics,” can introduce a noise component to the measurement of the reference and sample energy beams Eᵣ, Eₛ using some pyroelectric infrared detectors. Because it may be desirable for the analyzer 1210 to provide high signal-to-noise ratio measurements, such as, e.g., S/N in excess of 100 dB, some embodiments of the analyzer 1210 utilize one or more vibrational noise reduction apparatus or methods. For example, the analyzer 1210 may be mechanically isolated so that high S/N spectroscopic measurements can be obtained for vibrations below an acceleration of about 1.5 G.

In some embodiments of the analyzer 1210, vibrational noise can be reduced by using a temporally modulated energy source 1212 combined with an output filter. In some embodiments, the energy source 1212 is modulated at a known source frequency, and measurements made by the detectors 1236 and 1230 are filtered using a narrowband filter centered at the source frequency. For example, in some embodiments, the energy output of the source 1212 is sinusoidally modulated at 10 Hz, and outputs of the detectors 1236 and 1230 are filtered using a narrow bandpass filter of less than about 1 Hz centered at 10 Hz. Accordingly, microphonic signals that are not at 10 Hz are significantly attenuated. In some embodiments, the modulation depth of the energy beam E may be greater than 50% such as, for example, 80%. The duty cycle of the beam may be between about 30% and 70%. The temporal modulation may be sinusoidal or any other waveform. In embodiments utilizing temporally modulated energy sources, detector output may be filtered using a synchronous demodulator and digital filter. The demodulator and filter are software components that may be digitally implemented in a processor such as the algorithm processor 416. Synchronous demodulators, coupled with low pass filters, are often referred to as “lock in amplifiers.”

The analyzer 1210 may also include a vibration sensor 1232 (e.g., one or more accelerometers) disposed near one (or both) of the detectors 1236 and 1230. The output of the vibration sensor 1232 is monitored, and suitable actions are taken if the measured vibration exceeds a vibration threshold. For example, in some embodiments, if the vibration sensor 1232 detects above-threshold vibrations, the system discards any ongoing measurement and “holds off” on performing further measurements until the vibrations drop below the threshold. Discarded measurements may be repeated after the vibrations drop below the vibration threshold. In some embodiments, if the duration of the “hold off” is sufficiently long, the fluid in the sample cell 1230 is flushed, and a new fluid sample is delivered to the cell 1230 for measurement. The vibration threshold may be selected so that the error in analyte measurement is at an acceptable level for vibrations below the threshold. In some embodiments, the threshold corresponds to an error in glucose concentration of 5 mg/dL. The vibration threshold may be determined individually for each filter 1215.

Certain embodiments of the analyzer 1210 include a temperature system (not shown in FIG. 12) for monitoring and/or regulating the temperature of system components (such as the detectors 1236, 1230) and/or the fluid sample. Such a temperature system may include temperature sensors, thermoelectrical heat pumps (e.g., a Peltier device), and/or thermistors, as well as a control system for monitoring and/or regulating temperature. In some embodiments, the control system comprises a proportional-plus-integral-plus-derivative (PID) control. For example, in some embodiments, the temperature system is used to regulate the temperature of the detectors 1230, 1236 to a desired operating temperature, such as 35 degrees Celsius.

Optical Measurement

The analyzer 1210 illustrated in FIG. 12 can be used to determine optical properties of a substance in the sample cell 1248. The substance may include whole blood, plasma, saline, water, air or other substances. In some embodiments, the optical properties include measurements of an absorbance, transmittance, and/or optical density in the wavelength passbands of some or all of the filters 1215 disposed in the filter wheel 1218. As described above, a measurement cycle comprises disposing one or more filters 1215 in the energy beam E for a dwell time and measuring a reference signal with the reference detector 1236 and a sample signal with the sample detector 1230. The number of filters 1215 used in the measurement cycle will be denoted by N, and each filter 1215 passes energy in a passband around a center wavelength λ, where i is an index ranging over the number of filters (e.g., from 1 to N). The set of optical measurements from the sample detector 1236 in the passbands of the N filters 1215 provide a wavelength-dependent spectrum of the substance in the sample cell 1248. The spectrum will be denoted by Cₛ(λᵢ), where Cₛ may be a transmittance, absorbance, optical density, or some other measure of an optical property of the substance. In some embodiments, the spectrum is normalized with respect to one or more of the reference signals measured by the reference detector 1230 and/or with respect to spectra of a reference substance (e.g., air or saline). The measured spectra are communicated to the algorithm processor 416 for calculation of the concentration of the analyte(s) of interest in the fluid sample.

In some embodiments, the analyzer 1210 performs spectroscopic measurements on the fluid sample (known as a “wet” reading) and on one or more reference samples. For example, an “air” reading occurs when the sample detector 1236 measures the sample signal without the sample cell 1248 in place along the optical axis X. (This can occur, for example, when the opposite opening 1030 is aligned with the optical axis X). A “water” or “saline” reading occurs when the sample cell 1248 is filled with water or saline, respectively. The algorithm processor 416 may be programmed to calculate analyte concentration using a combination of these spectral measurements.

In some embodiments, a pathlength corrected spectrum is calculated using wet, air, and reference readings. For example, the transmittance at wavelength λᵢ, denoted by Tᵢ, may be calculated according to Tᵢ=(Sᵢ(wet)/Rᵢ(wet))/(Sᵢ(air)/Rᵢ(air)), where Sᵢ denotes the sample signal from the sample detector 1236 and Rᵢ denotes the corresponding reference signal from the reference detector 1230. In some embodiments, the algorithm processor 416 calculates the optical density, ODᵢ, as a logarithm of the transmittance, e.g., according to ODᵢ=−Log(Tᵢ). In one implementation, the analyzer 1210 takes a set of wet readings in each of the N filter passbands and then takes a set of air readings in each of the N filter passbands. In other embodiments, the analyzer 1210 may take an air reading before (or after) the corresponding wet reading.

The optical density ODᵢ is the product of the absorption coefficient at wavelength λᵢ, αᵢ, times the pathlength L over which the sample energy beam Eₛ interacts with the substance in the sample chamber 1248, e.g., ODᵢ=αᵢ L. The absorption coefficient αᵢ of a substance may be written as the product of an absorptivity per mole times a molar concentration of the substance. FIG. 12 schematically illustrates the pathlength L of the sample cell 1248. The pathlength L may be determined from spectral measurements made when the sample cell 1248 is filled with a reference substance. For example, because the absorption coefficient for water (or saline) is known, one or more water (or saline) readings can be used to determine the pathlength L from measurements of the transmittance (or optical density) through the cell 1248. In some embodiments, several readings are taken in different wavelength passbands, and a curve-fitting procedure is used to estimate a best-fit pathlength L. The pathlength L may be estimated using other methods including, for example, measuring interference fringes of light passing through an empty sample cell 1248.

The pathlength L may be used to determine the absorption coefficients of the fluid sample at each wavelength. Molar concentration of an analyte of interest can be determined from the absorption coefficient and the known molar absorptivity of the analyte. In some embodiments, a sample measurement cycle comprises a saline reading (at one or more wavelengths), a set of N wet readings (taken, for example, through a sample cell 1248 containing saline solution), followed by a set of N air readings (taken, for example, through the opposite opening 1030). As discussed above, the sample measurement cycle can be performed in about 2 minutes when the filter dwell times are about 2 seconds. After the sample measurement cycle is completed, a detergent cleaner may be flushed through the sample holder 1248 to reduce buildup of organic matter (e.g., proteins) on the windows of the sample holder 1248. The detergent is then flushed to a waste bladder.

In some embodiments, the system stores information related to the spectral measurements so that the information is readily available for recall by a user. The stored information may include wavelength-dependent spectral measurements (including fluid sample, air, and/or saline readings), computed analyte values, system temperatures and electrical properties (e.g., voltages and currents), and any other data related to use of the system (e.g., system alerts, vibration readings, S/N ratios, etc.). The stored information may be retained in the system for a time period such as, for example, 30 days. After this time period, the stored information may be communicated to an archival data storage system and then deleted from the system. In some embodiments, the stored information is communicated to the archival data storage system via wired or wireless methods, e.g., over a hospital information system (HIS).

Algorithm

The algorithm processor 416 (FIG. 4) (or any other suitable processor) may be configured to receive from the analyzer 1210 the wavelength-dependent optical measurements Cs(λᵢ) of the fluid sample. In some embodiments, the optical measurements comprise spectra such as, for example, optical densities ODᵢ measured in each of the N filter passbands centered around wavelengths λᵢ. The optical measurements Cs(λᵢ) are communicated to the processor 416, which analyzes the optical measurements to detect and quantify one or more analytes in the presence of interferents. In some embodiments, one or more poor quality optical measurements Cs(λᵢ) are rejected (e.g., as having a S/N ratio that is too low), and the analysis performed on the remaining, sufficiently high-quality measurements. In another embodiment, additional optical measurements of the fluid sample are taken by the analyzer 1210 to replace one or more of the poor quality measurements.

Interferents can comprise components of a material sample being analyzed for an analyte, where the presence of the interferent affects the quantification of the analyte. Thus, for example, in the spectroscopic analysis of a sample to determine an analyte concentration, an interferent could be a compound having spectroscopic features that overlap with those of the analyte, in at least a portion of the wavelength range of the measurements. The presence of such an interferent can introduce errors in the quantification of the analyte. More specifically, the presence of one or more interferents can affect the sensitivity of a measurement technique to the concentration of analytes of interest in a material sample, especially when the system is calibrated in the absence of, or with an unknown amount of, the interferent.

Independently of or in combination with the attributes of interferents described above, interferents can be classified as being endogenous (i.e., originating within the body) or exogenous (i.e., introduced from or produced outside the body). As an example of these classes of interferents, consider the analysis of a blood sample (or a blood component sample or a blood plasma sample) for the analyte glucose. Endogenous interferents include those blood components having origins within the body that affect the quantification of glucose, and may include water, hemoglobin, blood cells, and any other component that naturally occurs in blood. Exogenous interferents include those blood components having origins outside of the body that affect the quantification of glucose, and can include items administered to a person, such as medicaments, drugs, foods or herbs, whether administered orally, intravenously, topically, etc.

Independently of or in combination with the attributes of interferents described above, interferents can comprise components which are possibly, but not necessarily, present in the sample type under analysis. In the example of analyzing samples of blood or blood plasma drawn from patients who are receiving medical treatment, a medicament such as acetaminophen is possibly, but not necessarily, present in this sample type. In contrast, water is necessarily present in such blood or plasma samples.

Certain disclosed analysis methods are particularly effective if each analyte and interferent has a characteristic signature in the measurement (e.g., a characteristic spectroscopic feature), and if the measurement is approximately affine (e.g., includes a linear term and an offset) with respect to the concentration of each analyte and interferent. In such methods, a calibration process is used to determine a set of one or more calibration coefficients and a set of one or more optional offset values that permit the quantitative estimation of an analyte. For example, the calibration coefficients and the offsets may be used to calculate an analyte concentration from spectroscopic measurements of a material sample (e.g., the concentration of glucose in blood plasma). In some of these methods, the concentration of the analyte is estimated by multiplying the calibration coefficient by a measurement value (e.g., an optical density) to estimate the concentration of the analyte. Both the calibration coefficient and measurement can comprise arrays of numbers. For example, in some embodiments, the measurement comprises spectra Cₛ(λᵢ) measured at the wavelengths λᵢ, and the calibration coefficient and optional offset comprise an array of values corresponding to each wavelength λᵢ. In some embodiments, as further described below, a hybrid linear analysis (HLA) technique is used to estimate analyte concentration in the presence of a set of interferents, while retaining a high degree of sensitivity to the desired analyte. The data used to accommodate the set of possible interferents may include (a) signatures of each of the members of the family of potential additional substances and (b) a typical quantitative level at which each additional substance, if present, is likely to appear. In some embodiments, the calibration coefficient (and optional offset) are adjusted to minimize or reduce the sensitivity of the calibration to the presence of interferents that are identified as possibly being present in the fluid sample.

In some embodiments, the analyte analysis method uses a set of training spectra each having known analyte concentration and produces a calibration that minimizes the variation in estimated analyte concentration with interferent concentration. The resulting calibration coefficient indicates sensitivity of the measurement to analyte concentration. The training spectra need not include a spectrum from the individual whose analyte concentration is to be determined. That is, the term “training” when used in reference to the disclosed methods does not require training using measurements from the individual whose analyte concentration will be estimated (e.g., by analyzing a bodily fluid sample drawn from the individual).

Several terms are used herein to describe the analyte analysis process. The term “Sample Population” is a broad term and includes, without limitation, a large number of samples having measurements that are used in the computation of calibration values (e.g., calibration coefficients and optional offsets). The samples may be used to train the method of generating calibration values. For an embodiment involving the spectroscopic determination of glucose concentration, the Sample Population measurements can each include a spectrum (analysis measurement) and a glucose concentration (analyte measurement). In some embodiments, the Sample Population measurements are stored in a database, referred to herein as a “Population Database.”

The Sample Population may or may not be derived from measurements of material samples that contain interferents to the measurement of the analyte(s) of interest. One distinction made herein between different interferents is based on whether the interferent is present in both the Sample Population and the particular sample being measured, or only in the sample. As used herein, the term “Type-A interferent” refers to an interferent that is present in both the Sample Population and in the material sample being measured to determine an analyte concentration. In certain methods, the Sample Population includes interferents that are endogenous, and generally does not include exogenous interferents, and thus the Type-A interferents are generally endogenous. The number of Type-A interferents depends on the measurement and analyte(s) of interest, and may number, in general, from zero to a very large number (e.g., greater than 300). All of the Type-A interferents typically are not expected to be present in a particular material sample, and in many cases, a smaller number of interferents (e.g., 5, 10, 15, 20, or 25) may be used in the analysis. In certain embodiments, the number of interferents used in the analysis is less than or equal to the number of wavelength-dependent measurements N in the spectrum Cs(λᵢ).

The material sample being measured, for example a fluid sample in the sample cell 1248, may also include interferents that are not present in the Sample Population. As used herein, the term “Type-B interferent” refers to an interferent that is either: 1) not found in the Sample Population but that is found in the material sample being measured (e.g., an exogenous interferent), or 2) is found naturally in the Sample Population, but is at abnormal concentrations (e.g. high or low) in the material sample (e.g., an endogenous interferent). Examples of a Type-B exogenous interferent may include medications, and examples of Type-B endogenous interferents may include urea in persons suffering from renal failure. For example, in mid-infrared spectroscopic absorption measurements of glucose in blood (or blood plasma), water is present in all fluid samples, and is thus a Type-A interferent. For a Sample Population made up of individuals who are not taking intravenous drugs, and a material sample taken from a hospital patient who is being administered a selected intravenous drug, the selected drug is a Type-B interferent. In addition to components naturally found in the blood, the ingestion or injection of some medicines or illicit drugs can result in very high and rapidly changing concentrations of exogenous interferents.

In some embodiment, a list of one or more possible Type-B Interferents is referred to herein as forming a “Library of Interferents,” and each interferent in the library is referred to as a “Library Interferent.” The Library Interferents include exogenous interferents and endogenous interferents that may be present in a material sample due, for example, to a medical condition causing abnormally high concentrations of the endogenous interferent.

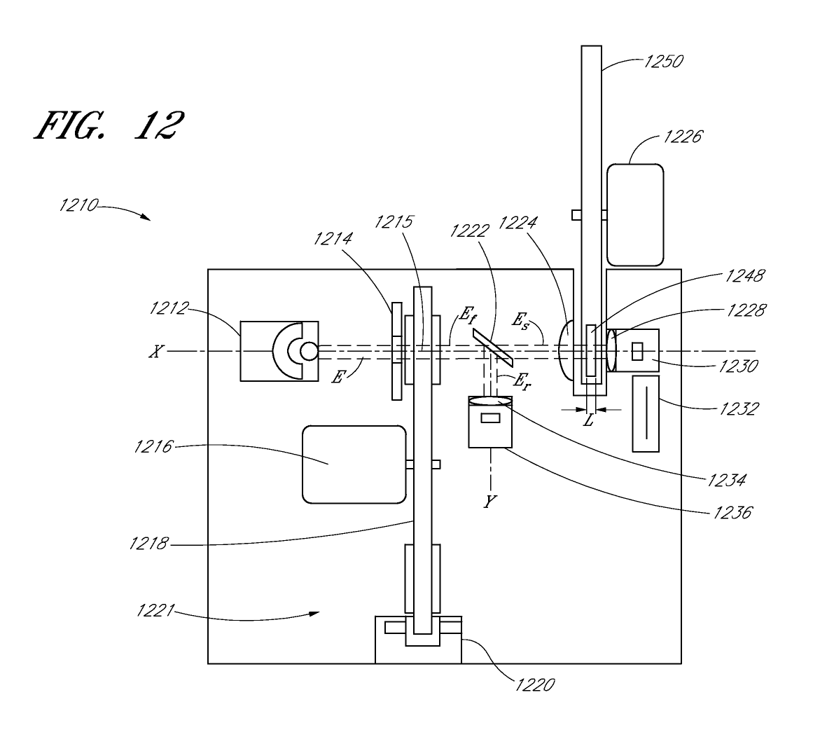

FIG. 13 is a flowchart that schematically illustrates an embodiment of a method 1300 for estimating the concentration of an analyte in the presence of interferents. In block 1310, a measurement of a sample is obtained, and in block 1320 data relating to the obtained measurement is analyzed to identify possible interferents to the analyte. In block 1330, a model is generated for predicting the analyte concentration in the presence of the identified possible interferents, and in block 1340 the model is used to estimate the analyte concentration in the sample from the measurement. In certain embodiments of the method 1300, the model generated in block 1330 is selected to reduce or minimize the effect of identified interferents that are not present in a general population of which the sample is a member.

An example embodiment of the method 1300 of FIG. 13 for the determination of an analyte (e.g., glucose) in a blood sample will now be described. This example embodiment is intended to illustrate various aspects of the method 1300 but is not intended as a limitation on the scope of the method 1300 or on the range of possible analytes. In this example, the sample measurement in block 1310 is an absorption spectrum, Cs(λᵢ), of a measurement sample S that has, in general, one analyte of interest, glucose, and one or more interferents. In general, the sample S includes Type-A interferents, at concentrations preferably within the range of those found in the Sample Population.

In block 1320, a statistical comparison of the absorption spectrum of the sample S with a spectrum of the Sample Population and combinations of individual Library Interferent spectra is performed. The statistical comparison provides a list of Library Interferents that are possibly contained in sample S and may include either no Library Interferents or one or more Library Interferents. In this example, in block 1330, one or more sets of spectra are generated from spectra of the Sample Population and their respective known analyte concentrations and known spectra of the Library Interferents identified in block 1320. In block 1330, the generated spectra are used to calculate a model for predicting the analyte concentration from the obtained measurement. In some embodiments, the model comprises one or more calibration coefficients κ(λᵢ) that can be used with the sample measurements Cs(λᵢ) to provide an estimate of the analyte concentration, gₑₛₜ. In block 1340, the estimated analyte concentration is determined form the model generated in block 1330. For example, in some embodiments of HLA, the estimated analyte concentration is calculated according to a linear formula: gₑₛₜ=κ(λᵢ)·Cₛ(λᵢ). Because the absorption measurements and calibration coefficients may represent arrays of numbers, the multiplication operation indicated in the preceding formula may comprise a sum of the products of the measurements and coefficients (e.g., an inner product or a matrix product). In some embodiments, the calibration coefficient is determined so as to have reduced or minimal sensitivity to the presence of the identified Library Interferents.

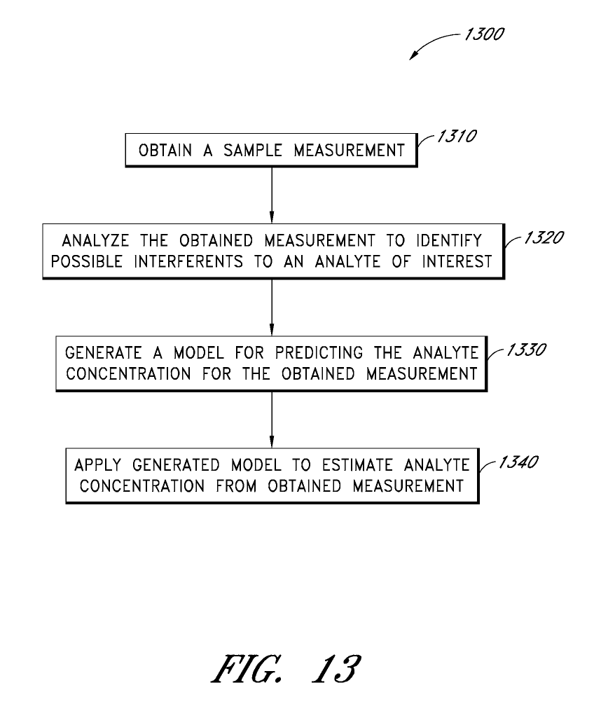

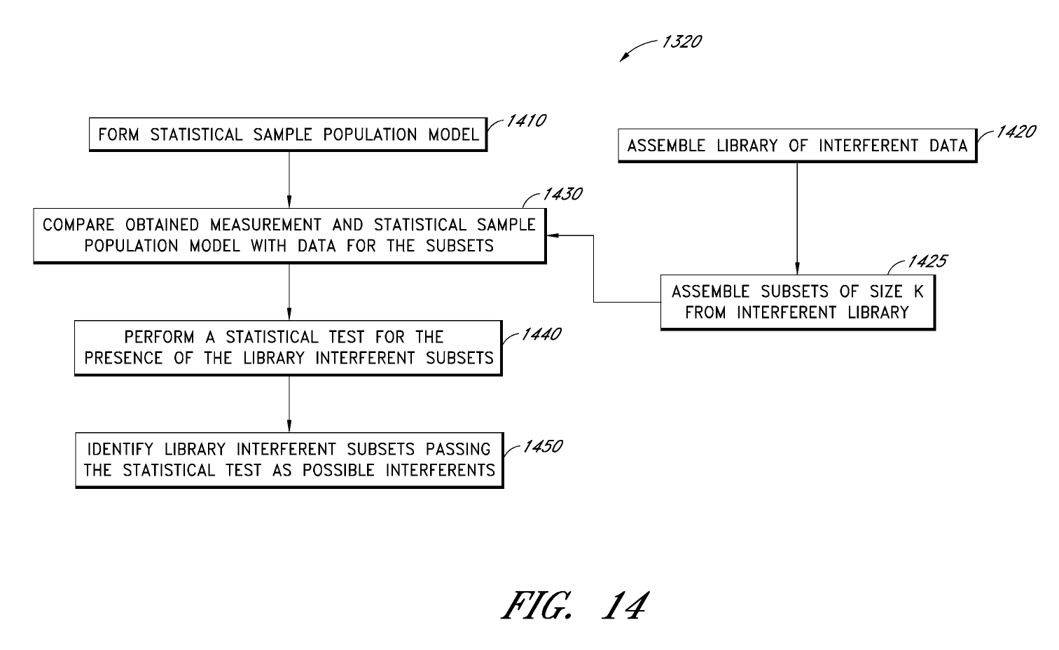

An example embodiment of block 1320 of the method 1300 will now be described with reference to FIG. 14. In this example, block 1320 includes forming a statistical Sample Population model (block 1410), assembling a library of interferent data (block 1420), assembling all subsets of size K of the library interferents (block 1425), comparing the obtained measurement and statistical Sample Population model with data for each set of interferents from an interferent library (block 1430), performing a statistical test for the presence of each interferent from the interferent library (block 1440), and identifying possible interferents that pass the statistical test (block 1450). The size K of the subsets may be an integer such as, for example, 1, 2, 3, 4, 5, 6, 10, 16, or more. The acts of block 1420 can be performed once or can be updated as necessary. In certain embodiments, the acts of blocks 1430, 1440, and 1450 are performed sequentially for all subsets of Library Interferents that pass the statistical test (block 1440).

In this example, in block 1410, a Sample Population Database is formed that includes a statistically large Sample Population of individual spectra taken over the same wavelength range as the sample spectrum, Cₛ(λᵢ). The Database also includes an analyte concentration corresponding to each spectrum. For example, if there are P Sample Population spectra, then the spectra in the Database can be represented as C={C₁, C₂, . . . , C_{P}}, and the analyte concentration corresponding to each spectrum can be represented as g={g₁, g₂, . . . , g_{P}}. In some embodiments, the Sample Population does not have any of the Library Interferents present, and the material sample has interferents contained in the Sample Population and one or more of the Library Interferents. Stated in terms of Type-A and Type-B interferents, the Sample Population has Type-A interferents, and the material sample has Type-A and may have Type-B interferents.

In some embodiments of block 1410, the statistical sample model comprises a mean spectrum and a covariance matrix calculated for the Sample Population. For example, if each spectrum measured at N wavelengths λᵢ is represented by an N×1 array, C, then the mean spectrum, μ, is an N×1 array having values at each wavelength averaged over the range of spectra in the Sample Population. The covariance matrix, V, is calculated as the expected value of the deviation between C and μ and can be written as V=E((C−μ)(C−μ)^{T}) where E(·) represents the expected value and the superscript T denotes transpose. In other embodiments, additional statistical parameters may be included in the statistical model of the Sample Population spectra.

Additionally, a Library of Interferents may be assembled in block 1420. A number of possible interferents can be identified, for example, as a list of possible medications or foods that might be ingested by the population of patients at issue. Spectra of these interferents can be obtained, and a range of expected interferent concentrations in the blood, or other expected sample material, can be estimated. In certain embodiments, the Library of Interferents includes, for each of “M” interferents, the absorption spectrum of each interferent, IF={IF₁, IF₂, . . . , IF_{M}}, and a range of concentrations for each interferent from Tmax={Tmax₁, Tmax₂, . . . , Tmax_{M}) to Tmin={Tmin₁, Tmin₂, . . . , Tmin_{M}). Information in the Library may be assembled once and accessed as needed. For example, the Library and the statistical model of the Sample Population may be stored in a storage device associated with the algorithm processor 416 (see, FIG. 4).

Continuing in block 1425, the algorithm processor 416 assembles one or more subsets comprising a number K of spectra taken from the Library of Interferents. The number K may be an integer such as, for example, 1, 2, 3, 4, 5, 6, 10, 16, or more. In some embodiments, the subsets comprise all combinations of the M Library spectra taken K at a time. In these embodiments, the number of subsets having K spectra is M!/(K!(M−K)!), where ! represents the factorial function.

Continuing in block 1430, the obtained measurement data (e.g., the sample spectrum) and the statistical Sample Population model (e.g., the mean spectrum and the covariance matrix) are compared with data for each subset of interferents determined in block 1425 in order to determine the presence of possible interferents in the sample (block 1440). In some embodiments, the statistical test for the presence of an interferent subset in block 1440 comprises determining the concentrations of each subset of interferences that minimize a statistical measure of “distance” between a modified spectrum of the material sample and the statistical model of the Sample Population (e.g., the mean μ and the covariance V). The concentrations may be calculated numerically. In some embodiments, the concentrations are calculated by algebraically solving a set of linear equations. The statistical measure of distance may comprise the well-known Mahalanobis distance (or Mahalanobis distance squared) and/or some other suitable statistical distance metric (e.g., Hotelling's T-square statistic). In certain implementations, the modified spectrum is given by C′ₛ(T)=Cₛ−IF·T where T=(T₁, T₂, . . . T_{K}) is a K-dimensional vector of interferent concentrations and IF={IF₁, IF₂, . . . IF_{K}} represents the K interferent absorption spectra of the subset (each normalized to have unit interferent concentration). In some embodiments, concentration of the i^{th} interferent is assumed to be in a range from a minimum value, Tminᵢ, to a maximum value, Tmaxᵢ. The value of Tminᵢ may be zero, or may be a value between zero and Tmaxᵢ, such as a fraction of Tmaxᵢ, or may be a negative value. Negative values represent interferent concentrations that are smaller than baseline interferent values in the Sample Population.

In block 1450, a list of possible interferent subsets ξ may be identified as the particular subsets that pass one or more statistical tests (in block 1440) for being present in the material sample. One or more statistical tests may be used, alone or in combination, to identify the possible interferents. For example, if a statistical test indicates that an i^{th} interferent is present in a concentration outside the range Tminᵢ to Tmaxᵢ, then this result may be used to exclude the i^{th} interferent from the list of possible interferents. In some embodiments, only the single most probable interferent subset is included on the list, for example, the subset having the smallest statistical distance (e.g., Mahalanobis distance). In an embodiment, the list includes the subsets ξ having statistical distances smaller than a threshold value. In certain embodiments, the list includes a number N_{S} of subsets having the smallest statistical distances, e.g., the list comprises the “best” candidate subsets. The number N_{S} may be any suitable integer such as 10, 20, 50, 100, 200, or more. An advantage of selecting the “best” N_{S} subsets is reduced computational burden on the algorithm processor 416. In certain such embodiments, the list is selected to comprise combinations of the N_{S} subsets taken L at a time. For example, in some embodiments, pairs of subsets are taken (e.g., L=2). An advantage of selecting pairs of subsets is that pairing captures the most likely combinations of interferents and the “best” candidates are included multiple times in the list of possible interferents. In embodiments in which combinations of L subsets are selected, the number of combinations of subsets in the list of possible interferent subsets is N_{S}!/(L!(N_{S}−L)!).

In other embodiments, the list of possible interferent subsets ξ is determined using a combination of some or all of the above criteria. In another embodiment, the list of possible interferent subsets ξ includes each of the subsets assembled in block 1425. A skilled artisan will recognize that many selection criteria are possible for the list of possible interferent subsets ξ

Returning to FIG. 13, the method 1300 continues in block 1330 where analyte concentration is estimated in the presence of the possible interferent subsets ξ determined in block 1450. FIG. 15 is a flowchart that schematically illustrates an example embodiment of the acts of block 1330. In block *O10, synthesized Sample Population measurements are generated to form an Interferent Enhanced Spectral Database (IESD). In block *O60, the IESD and known analyte concentrations are used to generate calibration coefficients for the selected interferent subset. As indicated in block *O65, blocks *O10 and *O60 may be repeated for each interferent subset ξ identified in the list of possible interferent subsets (e.g., in block 1450 of FIG. 14). In this example embodiment, when all the interferent subsets ξ have been processed, the method continues in block *O70, wherein an average calibration coefficient is applied to the measured spectra to determine a set of analyte concentrations.

In one example embodiment for block *O10, synthesized Sample Population spectra are generated by adding random concentrations of each interferent in one of the possible interferent subsets ξ. These spectra are referred to herein as an Interferent-Enhanced Spectral Database or IESD. In one example method, the IESD is formed as follows. A plurality of Randomly-Scaled Single Interferent Spectra (RSIS) are formed for each interferent in the interferent subset ξ. Each RSIS is formed by combinations of the interferent having spectrum IF multiplied by the maximum concentration Tmax, which is scaled by a random factor between zero and one. In certain embodiments, the scaling places the maximum concentration at the 95^{th} percentile of a log-normal distribution in order to generate a wide range of concentrations. In some embodiments, the log-normal distribution has a standard deviation equal to half of its mean value.

In this example method, individual RSIS are then combined independently and in random combinations to form a large family of Combination Interferent Spectra (CIS), with each spectrum in the CIS comprising a random combination of RSIS, selected from the full set of identified Library Interferents. An advantage of this method of selecting the CIS is that it produces adequate variability with respect to each interferent, independently across separate interferents.

The CIS and replicates of the Sample Population spectra are combined to form the IESD. Since the interferent spectra and the Sample Population spectra may have been obtained from measurements having different optical pathlengths, the CIS may be scaled to the same pathlength as the Sample Population spectra. The Sample Population Database is then replicated R times, where R depends on factors including the size of the Database and the number of interferents. The IESD includes R copies of each of the Sample Population spectra, where one copy is the original Sample Population Data, and the remaining R-1 copies each have one randomly chosen CIS spectra added. Accordingly, each of the IESD spectra has an associated analyte concentration from the Sample Population spectra used to form the particular IESD spectrum. In some embodiments, a 10-fold replication of the Sample Population Database is used for 130 Sample Population spectra obtained from 58 different individuals and 18 Library Interferents. A smaller replication factor may be used if there is greater spectral variety among the Library Interferent spectra, and a larger replication factor may be used if there is a greater number of Library Interferents.

After the IESD is generated in block *O10, in block *O60, the IESD spectra and the known, random concentrations of the subset interferents are used to generate a calibration coefficient for estimating the analyte concentration from a sample measurement. The calibration coefficient is calculated in some embodiments using a hybrid linear analysis (HLA) technique. In certain embodiments, the HLA technique includes constructing a set of spectra that are free of the desired analyte, projecting the analyte's spectrum orthogonally away from the space spanned by the analyte-free calibration spectra, and normalizing the result to produce a unit response. Further description of embodiments of HLA techniques may be found in, for example, “Measurement of Analytes in Human Serum and Whole Blood Samples by Near-Infrared Raman Spectroscopy,” Chapter 4, Andrew J. Berger, Ph. D. thesis, Massachusetts Institute of Technology, 1998, and “An Enhanced Algorithm for Linear Multivariate Calibration,” by Andrew J. Berger, et al., Analytical Chemistry, Vol. 70, No. 3, Feb. 1, 1998, pp. 623-627, the entirety of each of which is hereby incorporated by reference herein. A skilled artisan will recognize that in other embodiments the calibration coefficients may be calculated using other techniques including, for example, regression, partial least squares, and/or principal component analysis.

In block *O65, the processor 416 determines whether additional interferent subsets ξ remain in the list of possible interferent subsets. If another subset is present in the list, the acts in blocks *O10-*O60 are repeated for the next subset of interferents using different random concentrations. In some embodiments, blocks *O10-*O60 are performed for only the most probable subset on the list.

The calibration coefficient determined in block *O60 corresponds to a single interferent subset ξ from the list of possible interferent subsets and is denoted herein as a single-interferent-subset calibration coefficient κ_{avg}(ξ). In this example method, after all subsets ξ have been processed, the method continues in block *O70, in which the single-interferent-subset calibration coefficient is applied to the measured spectra Cₛ to determine an estimated, single-interferent-subset analyte concentration, g(ξ)=κ_{avg}(ξ)·Cₛ, for the interferent subset ξ. The set of the estimated, single-interferent-subset analyte concentrations g(ξ) for all subsets in the list may be assembled into an array of single-interferent-subset concentrations. As noted above, in some embodiments the blocks *O10-*O70 are performed once for the most probable single-interferent-subset on the list (e.g., the array of single-interferent analyte concentrations has a single member).

Returning to block 1340 of FIG. 13, the array of single-interferent-subset concentrations, g(ξ), is combined to determine an estimated analyte concentration, gₑₛₜ, for the material sample. In certain embodiments, a weighting function p(ξ) is determined for each of the interferent subsets ξ on the list of possible interferent subsets. The weighting functions may be normalized such that Σp(ξ)=1, where the sum is over all subsets ξ that have been processed from the list of possible interferent subsets. In some embodiments, the weighting functions can be related to the minimum Mahalanobis distance or an optimal concentration. In certain embodiments, the weighting function p(ξ), for each subset ξ, is selected to be a constant, e.g., 1N_{S} where N_{S} is the number of subsets processed from the list of possible interferent subsets. A person of ordinary skill will recognize that many different weighting functions p(ξ) can be selected.

In certain embodiments, the estimated analyte concentration, gₑₛₜ, is determined (in block 1340) by combining the single-interferent-subset estimates, g(ξ), and the weighting functions, p(ξ), to generate an average analyte concentration. The average concentration may be computed according to gₑₛₜ=Σg(ξ)p(ξ), where the sum is over the interferent subsets processed from the list of possible interferent subsets. In some embodiments, the weighting function p(ξ) is a constant value for each subset (e.g., a standard arithmetic average is used for determining average analyte concentration). By testing the above described example method on simulated data, it has been found that the average analyte concentration advantageously has reduced errors compared to other methods (e.g., methods using only a single most probable interferent).

User Interface

The system 400 may include a display system 414, for example, as depicted in FIG. 4. The display system 414 may comprise an input device including, for example, a keypad or a keyboard, a mouse, a touchscreen display, and/or any other suitable device for inputting commands and/or information. The display system 414 may also include an output device including, for example, an LCD monitor, a CRT monitor, a touchscreen display, a printer, and/or any other suitable device for outputting text, graphics, images, videos, etc. In some embodiments, a touchscreen display is advantageously used for both input and output.

The display system 414 may include a user interface 1600 by which users can conveniently and efficiently interact with the system 400. The user interface 1600 may be displayed on the output device of the system 400 (e.g., the touchscreen display).

FIGS. 16A and 16B schematically illustrate the visual appearance of embodiments of the user interface 1600. The user interface 1600 may show patient identification information 1602, which may include patient name and/or a patient ID number. The user interface 1600 also may include the current date and time 1604. An operating graphic 1606 shows the operating status of the system 400. For example, as shown in FIGS. 16A and 16B, the operating status is “Running,” which indicates that the system 400 is fluidly connected to the patient (“Jill Doe”) and performing normal system functions such as infusing fluid and/or drawing blood. The user interface 1600 can include one or more analyte concentration graphics 1608, 1612, which may show the name of the analyte and its last measured concentration. For example, the graphic 1608 in FIG. 16A shows “Glucose” concentration of 150 mg/dl, while the graphic 1612 shows “Lactate” concentration of 0.5 mmol/L. The particular analytes displayed and their measurement units (e.g., mg/dl, mmol/L, or other suitable unit) may be selected by the user. The size of the graphics 1608, 1612 may be selected to be easily readable out to a distance such as, e.g., 30 feet. The user interface 1600 may also include a next-reading graphic 1610 that indicates the time until the next analyte measurement is to be taken. In FIG. 16A, the time until next reading is 3 minutes, whereas in FIG. 16B, the time is 6 minutes, 13 seconds.

The user interface 1600 may include an analyte concentration status graphic 1614 that indicates status of the patient's current analyte concentration compared with a reference standard. For example, the analyte may be glucose, and the reference standard may be a hospital ICU's tight glycemic control (TGC). In FIG. 16A, the status graphic 1614 displays “High Glucose,” because the glucose concentration (150 mg/dl) exceeds the maximum value of the reference standard. In FIG. 16B, the status graphic 1614 displays “Low Glucose,” because the current glucose concentration (79 mg/dl) is below the minimum reference standard. If the analyte concentration is within bounds of the reference standard, the status graphic 1614 may indicate normal (e.g., “Normal Glucose”), or it may not be displayed at all. The status graphic 1614 may have a background color (e.g., red) when the analyte concentration exceeds the acceptable bounds of the reference standard.

The user interface 1600 may include one or more trend indicators 1616 that provide a graphic indicating the time history of the concentration of an analyte of interest. In FIGS. 16A and 16B, the trend indicator 1616 comprises a graph of the glucose concentration (in mg/dl) versus elapsed time (in hours) since the measurements started. The graph includes a trend line 1618 indicating the time-dependent glucose concentration. In other embodiments, the trend line 1618 may include measurement error bars and may be displayed as a series of individual data points. In FIG. 16B, the glucose trend indicator 1616 is shown as well as a trend indicator 1630 and trend line 1632 for the lactate concentration. In some embodiments, a user may select whether none, one, or both trend indicators 1616, 1618 are displayed. In some embodiments, one or both of the trend indicators 1616, 1618 may appear only when the corresponding analyte is in a range of interest such as, for example, above or below the bounds of a reference standard.

The user interface 1600 may include one or more buttons 1620-1626 that can be actuated by a user to provide additional functionality or to bring up suitable context-sensitive menus and/or screens. For example, in the embodiments shown in FIG. 16A and FIG. 16B, four buttons 1620-1626 are shown, although fewer or more buttons are used in other embodiments. The button 1620 (“End Monitoring”) may be pressed when one or more removable portions (see, e.g., 610 of FIG. 6) are to be removed. In many embodiments, because the removable portions 610, 612 are not reusable, a confirmation window appears when the button 1620 is pressed. If the user is certain that monitoring should stop, the user can confirm this by actuating an affirmative button in the confirmation window. If the button 1620 were pushed by mistake, the user can select a negative button in the confirmation window. If “End Monitoring” is confirmed, the system 400 performs appropriate actions to cease fluid infusion and blood draw and to permit ejection of a removable portion (e.g., the removable portion 610).

The button 1622 (“Pause”) may be actuated by the user if patient monitoring is to be interrupted but is not intended to end. For example, the “Pause” button 1622 may be actuated if the patient is to be temporarily disconnected from the system 400 (e.g., by disconnecting the tubes 306). After the patient is reconnected, the button 1622 may be pressed again to resume monitoring. In some embodiments, after the “Pause” button 1622 has been pressed, the button 1622 displays “Resume.”

The button 1624 (“Delay 5 Minutes”) causes the system 400 to delay the next measurement by a delay time period (e.g., 5 minutes in the depicted embodiments). Actuating the delay button 1624 may be advantageous if taking a reading would be temporarily inconvenient, for example, because a health care professional is attending to other needs of the patient. The delay button 1624 may be pressed repeatedly to provide longer delays. In some embodiments, pressing the delay button 1624 is ineffective if the accumulated delay exceeds a maximum threshold. The next-reading graphic 1610 automatically increases the displayed time until the next reading for every actuation of the delay button 1624 (up to the maximum delay).

The button 1626 (“Dose History”) may be actuated to bring up a dosing history window that displays patient dosing history for an analyte or medicament of interest. For example, in some embodiments, the dosing history window displays insulin dosing history of the patient and/or appropriate hospital dosing protocols. A nurse attending the patient can actuate the dosing history button 1626 to determine the time when the patient last received an insulin dose, the last dosage amount, and/or the time and amount of the next dosage. The system 400 may receive the patient dosing history via wired or wireless communications from a hospital information system.

In other embodiments, the user interface 1600 may include additional and/or different buttons, menus, screens, graphics, etc. that are used to implement additional and/or different functionalities.

Related Components

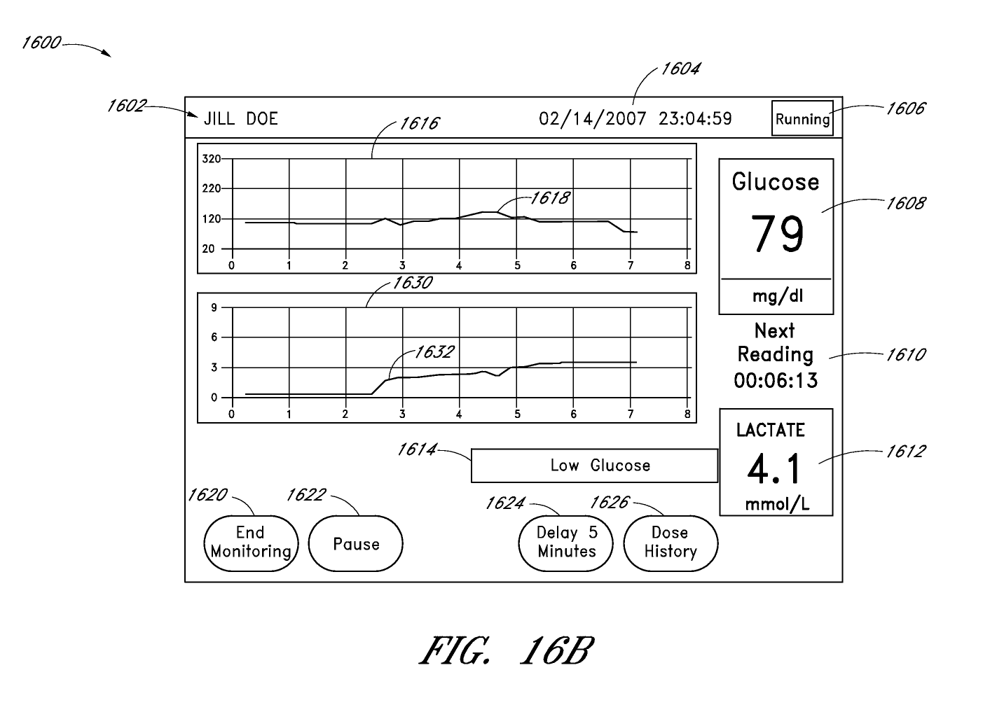

FIG. 17 schematically depicts various components and/or aspects of a patient monitoring system 17130 and how those components and/or aspects relate to each other. In some embodiments, the monitoring system 17130 can be the apparatus 100 for withdrawing and analyzing fluid samples. Some of the depicted components can be included in a kit containing a plurality of components. Some of the depicted components, including, for example, the components represented within the dashed rounded rectangle 17140 of FIG. 17, are optional and/or can be sold separately from other components.

The patient monitoring system 17130 shown in FIG. 17 includes a monitoring apparatus 17132. The monitoring apparatus 17132 can be the monitoring device 102, shown in FIG. 1 and/or the system 400 of FIG. 4. The monitoring apparatus 17132 can provide monitoring of physiological parameters of a patient. In some embodiments, the monitoring apparatus 17132 measures glucose and/or lactate concentrations in the patient's blood. In some embodiments, the measurement of such physiological parameters is substantially continuous. The monitoring apparatus 17132 may also measure other physiological parameters of the patient. In some embodiments, the monitoring apparatus 17132 is used in an intensive care unit (ICU) environment. In some embodiments, one monitoring apparatus 17132 is allocated to each patient room in an ICU. The patient monitoring system 17130 can include an optional interface cable 17142. In some embodiments, the interface cable 17142 connects the monitoring apparatus 17132 to a patient monitor (not shown). The interface cable 17142 can be used to transfer data from the monitoring apparatus 17132 to the patient monitor for display. In some embodiments, the patient monitor is a bedside cardiac monitor having a display that is located in the patient room (see, e.g., the user interface 1600 shown in FIG. 16A and FIG. 16B.) In some embodiments, the interface cable 17142 transfers data from the monitoring apparatus 17132 to a central station monitor and/or to a hospital information system (HIS). The ability to transfer data to a central station monitor and/or to a HIS may depend on the capabilities of the patient monitor system.

In the embodiment shown in FIG. 17, an optional bar code scanner 17144 is connected to the monitoring apparatus 17132. In some embodiments, the bar code scanner 17144 is used to enter patient identification codes, nurse identification codes, and/or other identifiers into the monitoring apparatus 17132. In some embodiments, the bar code scanner 17144 contains no moving parts. The bar code scanner 17144 can be operated by manually sweeping the scanner 17144 across a printed bar code or by any other suitable means. In some embodiments, the bar code scanner 17144 includes an elongated housing in the shape of a wand.

The patient monitoring system 17130 includes a fluid system kit 17134 connected to the monitoring apparatus 17132. In some embodiments, the fluid system kit 17134 includes fluidic tubes that connect a fluid source to an analytic subsystem. For example, the fluidic tubes can facilitate fluid communication between a blood source or a saline source and an assembly including a sample holder and/or a centrifuge. In some embodiments, the fluid system kit 17134 includes many of the components that enable operation of the monitoring apparatus 17132. In some embodiments, the fluid system kit 17134 can be used with anti-clotting agents (such as heparin), saline, a saline infusion set, a patient catheter, a port sharing IV infusion pump, and/or an infusion set for an IV infusion pump, any or all of which may be made by a variety of manufacturers. In some embodiments, the fluid system kit 17134 includes a monolithic housing that is sterile and disposable. In some embodiments, at least a portion of the fluid system kit 17134 is designed for single patient use. For example, the fluid system kit 17134 can be constructed such that it can be economically discarded and replaced with a new fluid system kit 17134 for every new patient to which the patient monitoring system 17130 is connected. In addition, at least a portion of the fluid system kit 17134 can be designed to be discarded after a certain period of use, such as a day, several days, several hours, three days, a combination of hours and days such as, for example, three days and two hours, or some other period of time. Limiting the period of use of the fluid system kit 17134 may decrease the risk of malfunction, infection, or other conditions that can result from use of a medical apparatus for an extended period of time.

In some embodiments, the fluid system kit 17134 includes a connector with a luer fitting for connection to a saline source. The connector may be, for example, a three-inch pigtail connector. In some embodiments, the fluid system kit 17134 can be used with a variety of spikes and/or IV sets used to connect to a saline bag. In some embodiments, the fluid system kit 17134 also includes a three-inch pigtail connector with a luer fitting for connection to one or more IV pumps. In some embodiments, the fluid system kit 17134 can be used with one or more IV sets made by a variety of manufacturers, including IV sets obtained by a user of the fluid system kit 17134 for use with an infusion pump. In some embodiments, the fluid system kit 17134 includes a tube with a low dead volume luer connector for attachment to a patient vascular access point. For example, the tube can be approximately seven feet in length and can be configured to connect to a proximal port of a cardiovascular catheter. In some embodiments, the fluid system kit 17134 can be used with a variety of cardiovascular catheters, which can be supplied, for example, by a user of the fluid system kit 17134. As shown in FIG. 17, the monitoring apparatus 17132 is connected to a support apparatus 17136, such as an IV pole. The support apparatus 17136 can be customized for use with the monitoring apparatus 17132. A vendor of the monitoring apparatus 17132 may choose to bundle the monitoring apparatus 17132 with a custom support apparatus 17136. In some embodiments, the support apparatus 17136 includes a mounting platform for the monitoring apparatus 17132. The mounting platform can include mounts that are adapted to engage threaded inserts in the monitoring apparatus 17132. The support apparatus 17136 can also include one or more cylindrical sections having a diameter of a standard IV pole, for example, so that other medical devices, such as IV pumps, can be mounted to the support apparatus. The support apparatus 17136 can also include a clamp adapted to secure the apparatus to a hospital bed, an ICU bed, or another variety of patient conveyance device.

In the embodiment shown in FIG. 17, the monitoring apparatus 17132 is electrically connected to an optional computer system 17146. The computer system 17146 can comprise one or multiple computers, and it can be used to communicate with one or more monitoring devices. In an ICU environment, the computer system 17146 can be connected to at least some of the monitoring devices in the ICU. The computer system 17146 can be used to control configurations and settings for multiple monitoring devices (for example, the system can be used to keep configurations and settings of a group of monitoring devices common). The computer system 17146 can also run optional software, such as data analysis software 17148, HIS interface software 17150, and insulin dosing software 17152.

In some embodiments, the computer system 17146 runs optional data analysis software 17148 that organizes and presents information obtained from one or more monitoring devices. In some embodiments, the data analysis software 17148 collects and analyzes data from the monitoring devices in an ICU. The data analysis software 17148 can also present charts, graphs, and statistics to a user of the computer system 17146.

In some embodiments, the computer system 17146 runs optional hospital information system (HIS) interface software 17150 that provides an interface point between one or more monitoring devices and an HIS. The HIS interface software 17150 may also be capable of communicating data between one or more monitoring devices and a laboratory information system (LIS).

In some embodiments, the computer system 17146 runs optional insulin dosing software 17152 that provides a platform for implementation of an insulin dosing regimen. In some embodiments, the hospital tight glycemic control protocol is included in the software. The protocol allows computation of proper insulin doses for a patient connected to a monitoring device 17146. The insulin dosing software 17152 can communicate with the monitoring device 17146 to ensure that proper insulin doses are calculated.

Accurate and Timely Body Fluid Analysis

Certain embodiments disclosed herein relate to a method and apparatus for determining the concentration of an analyte within a specified time frame, and more particularly to a method and system for measuring analytes, including but not limited to glucose, at concentrations useful for tight glycemic control of hospital patients, within 15 minutes or less.

One embodiment is directed to a device and method for measuring glucose within blood or other bodily fluid(s) with a standard error (STD) of 14 mg/dl or less. In one embodiment, the measurement is made, and a glucose concentration value preferably reported/displayed, within 25 minutes or less of initiating the draw of a fluid sample from a patient. In alternative embodiments, the measurement is made in one of the following time frames after having drawn a fluid sample from a patient: 24 minutes or less, 23 minutes or less, 22 minutes or less, 21 minutes or less, 20 minutes or less, 19 minutes or less, 18 minutes or less, 17 minutes or less, 16 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, 5 minutes or less, 4 minutes or less, 3 minutes or less, 2 minutes or less, 1 minute or less, 45 seconds or less, 30 seconds or less, or 15 seconds or less. In other alternative embodiments, the glucose measurement has a standard error (STD) of 13 mg/dl or less, 12 mg/dl or less, 11 mg/dl or less, 10 mg/dl or less, 9 mg/dl or less, 8 mg/dl or less, 7 mg/dl or less, 6 mg/dl or less, or 5 mg/dl or less.

Without limitation one preferred embodiment includes:
1. A full-time vascular connection to the patient
2. An automatic blood sampling apparatus
3. A built in plasma separation system to separate plasma from a blood sample and facilitate measurement of glucose concentration in the plasma
4. A rapid glucose analysis apparatus
5. A bedside readout of glucose concentration, e.g. a “real time” glucose concentration.

The sampling system 102 can perform measurements of glucose concentration with standard errors (STD) ranging from 14 mg/dl or less down to 5 mg/dl or less, at a repetition rate of from 25 minutes or less down to 15 seconds or less.

The following four examples present either actual data obtained from measurements on the blood of patients containing possible interferents or, where noted, results from simulations of the sampling system 102.

### Example 1

The sampling system 102 and its components can optionally be embodied as described in the discussion of this Example 1. The sampling system is a reagentless, continuous, point of care analyzer incorporating a Mid Infrared spectroscopic measurement engine and a single patient use centrifugal whole blood separator. Vascular access is made by direct connection to an arterial, central venous or peripheral venous catheter. The instrument automatically makes a plasma glucose measurement every 15 minutes using 40 microliters of whole blood per measurement. When used with a computational algorithm as set forth herein it is very well suited to rejecting the relatively large doses of injectable interferents and wide-ranging endogenous substances commonly found in the critical care setting.

A study was used to evaluate the baseline accuracy and the performance of Mid IR technology in actual ICU samples.

The sampling system used in the study includes a pole mounted, point of care, bedside monitor. It connects to a dedicated “bag” of saline which is used for KVO (keep vein open) infusion and system flushing. The sampling system also connects to a dedicated patient vascular line from which it automatically draws samples.

The “wetted” components are housed in a single patient use disposable. This disposable includes a combination flow cell and centrifuge as discussed elsewhere herein. A waste container in the disposable captures the 40 microliters of blood used for each measurement.

The sampling system uses Mid IR absorption measurements made at 25 fixed and specific Mid IR wavelengths with bandwidths from 0.2 micron to 0.35 micron and wavelengths from 4 to 12 microns. For this study, measurements were made on a laboratory spectrophotometer (Perkin Elmer FTIR). A software program reduced the continuous-spectrum data down to the 25 specified wavelengths before algorithm processing. The sampling system can optionally incorporate a fixed filter spectrophotometer (e.g., see the analyte detection system 1210 described in connection with FIG. 12), with one filter for each wavelength. The signal to noise ratio of the fixed filter spectrophotometer has been demonstrated to be superior to the laboratory spectrophotometer used in the study.

The study used Hybrid Linear Analysis (HLA) as described elsewhere herein to develop instrument calibration coefficients. Normal volunteer blood was collected, centrifuged to isolate the plasma component, doped to a wide range of glucose values and scanned. Using spectra resulting from these scans HLA methods were used to determine calibration coefficients. One coefficient was generated for each wavelength plus an offset term. The coefficients were unchanged throughout the prospective portions of the study.

HLA analysis proceeds through steps of obtaining spectra, performing a spectral quality check, checking for the presence of drugs, identifying any drugs that are present, and computing glucose concentration based in part on the drug presence and identification information. Spectral quality is ascertained regardless of drug or glucose content. If the quality is acceptable the process continues. If not the spectra is re-measured. The presence of a drug is identified using pre-determined measurement criteria. Measurements below the threshold cause the spectra to be sent directly to glucose computation step. Measurements above trigger an automatic algorithm adaptation, the first step of which is drug identification. Drug identification is accomplished using stored drug spectra and a series of computations. Calibration coefficients are adapted to accommodate the actual drugs on board. This minimizes correction magnitude and maximizes accuracy.

The first prospective evaluation of the sampling system used blood obtained from 21 normal, healthy volunteers. The blood was centrifuged to isolate the plasma component, the plasma was doped to various glucose levels and scanned. A program then applied the predetermined coefficients at the specific wavelengths to compute glucose values.

The second prospective evaluation of the sampling system used 318 samples of blood obtained from 94 patients admitted to the Stamford Hospital ICU in Stamford, Conn. The samples were separated to serum and frozen in the hospital laboratory before being shipped to an offsite laboratory where they were gamma sterilized, thawed and analyzed on a YSI 2700 reference laboratory analyzer as well as scanned by an FTIR device. The 25 wavelengths were used to analyze the serum for interferents. If the interferent detection algorithm indicated that the sample contained an interferent the interfering substance(s) was identified. Using pre-collected spectra of the pure interfering substance (which can be, in some embodiments, stored in the memory of the sampling system), the effect of that substance was reduced using an interferent rejection algorithm as discussed elsewhere herein. After the interferent removal process, the glucose concentration was computed using the 25 HLA-computed coefficients. In the study, 108 of the 318 ICU samples employed the interferent removal algorithm before computation of the glucose value.

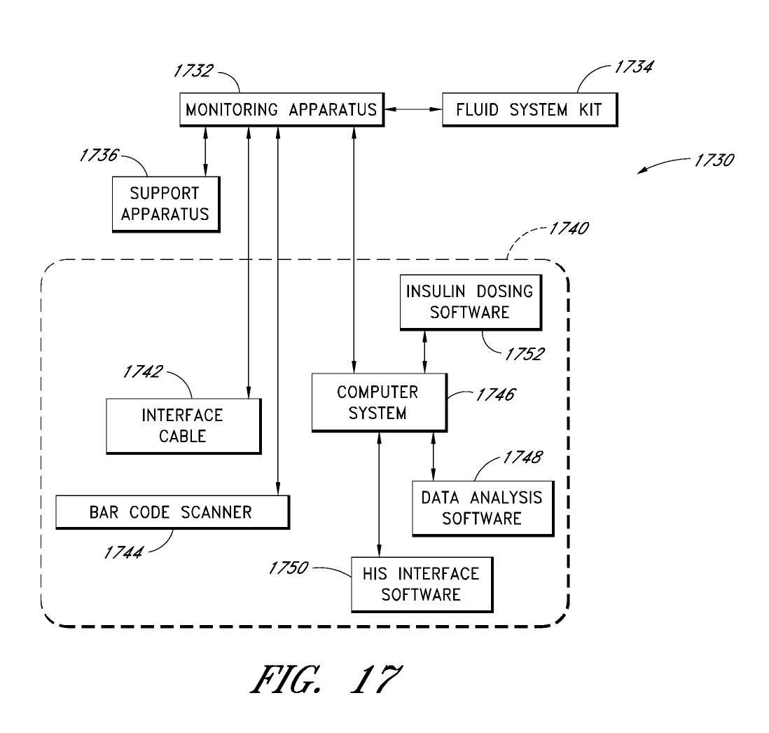

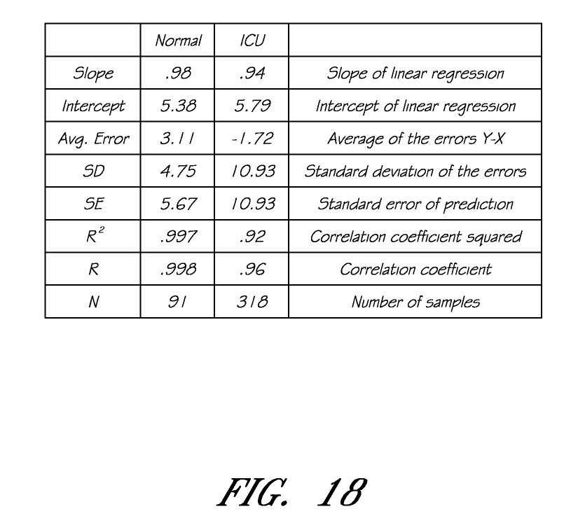

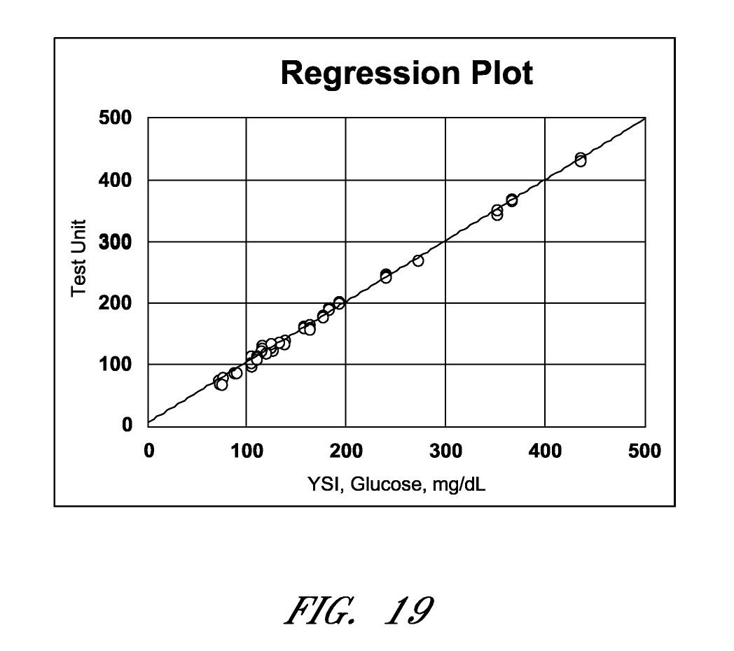

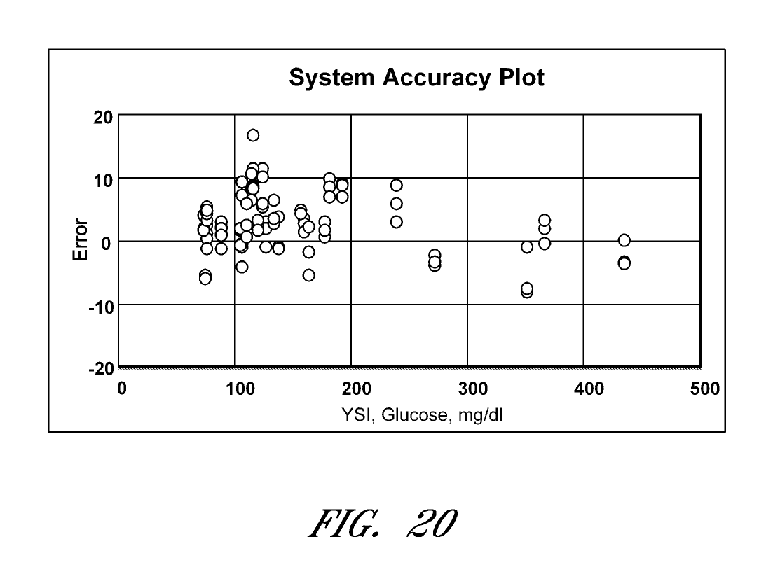

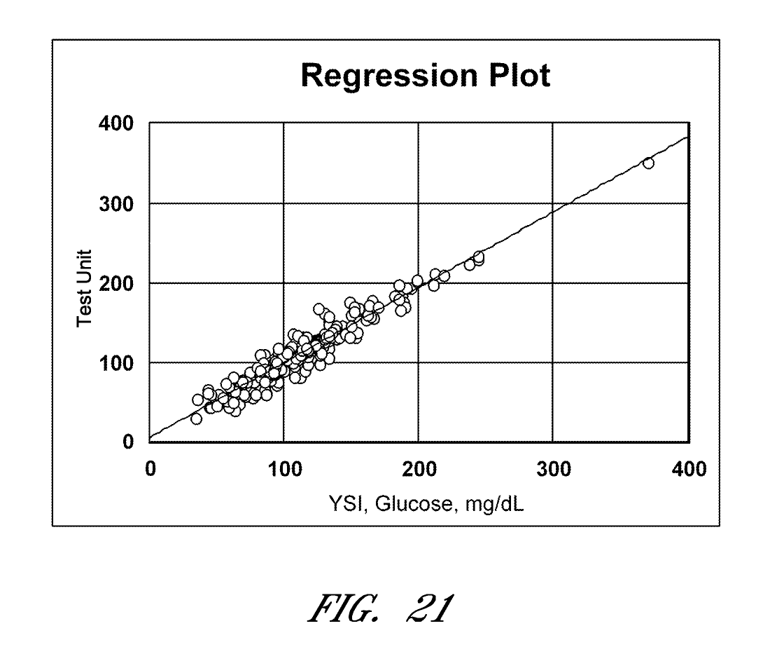

Performance metrics from the study can be seen in the table in FIG. 18, and in the graphs in FIGS. 19-22. Prospective measurement on Normal volunteers yielded a standard deviation of the errors (SD) of 4.7 mg/dL, with an R-squared of 0.997. Prospective measurement on ICU samples yielded an SD of 10.93 mg/dL, a standard error of prediction (SE) of 10.93 mg/dL, and with an R-squared of 0.92. The measurements were obtained with a spectrometer total integration time of 1 minute.

### Example 2

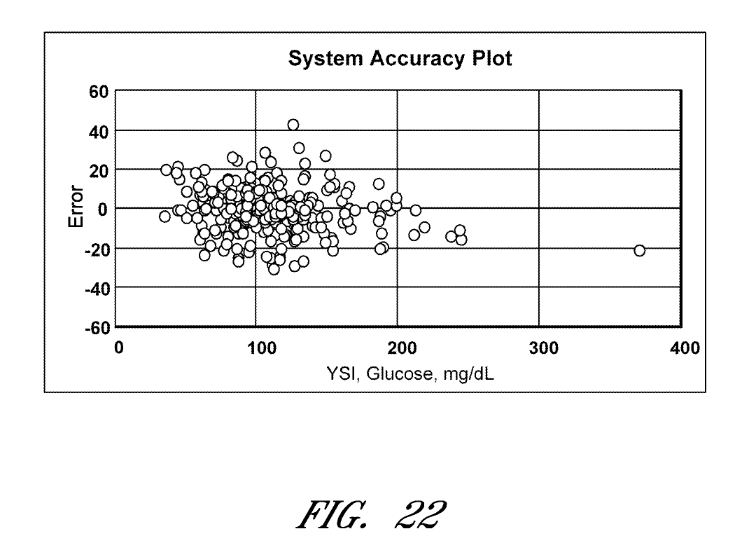

FIG. 23 shows a comparison of the results of measurements obtained with the sampling system (“Estimated”) of patients from an ICU at Stamford Hospital in Stamford, Conn. with measurements obtained with laboratory grade analytical equipment (“Reference”) of the Stamford ICU patients. The results show the effectiveness of the Interferent Rejection algorithm described herein on real blood samples, illustrating the standard error for the measurement of glucose, in the presence of interferents, to be 9.75 mg/dL with a spectrometer total integration time of 1 minute.

### Example 3

### Beta Performance Model Predictions

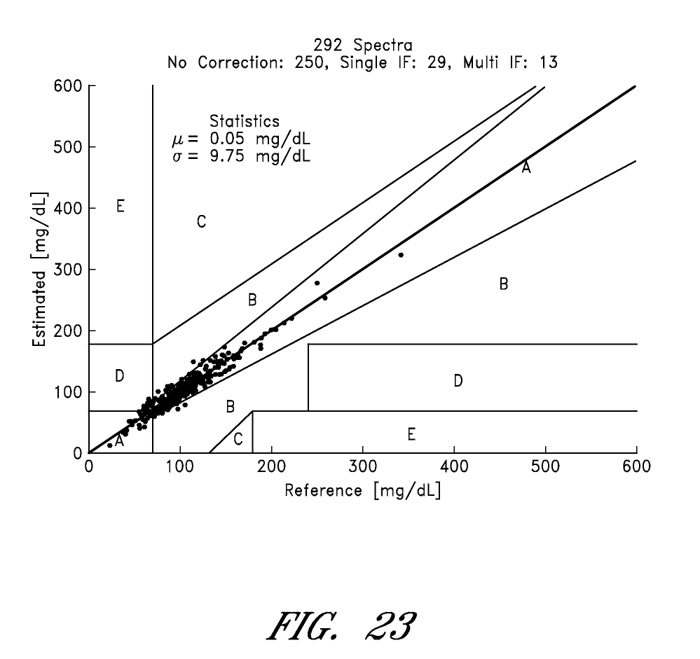

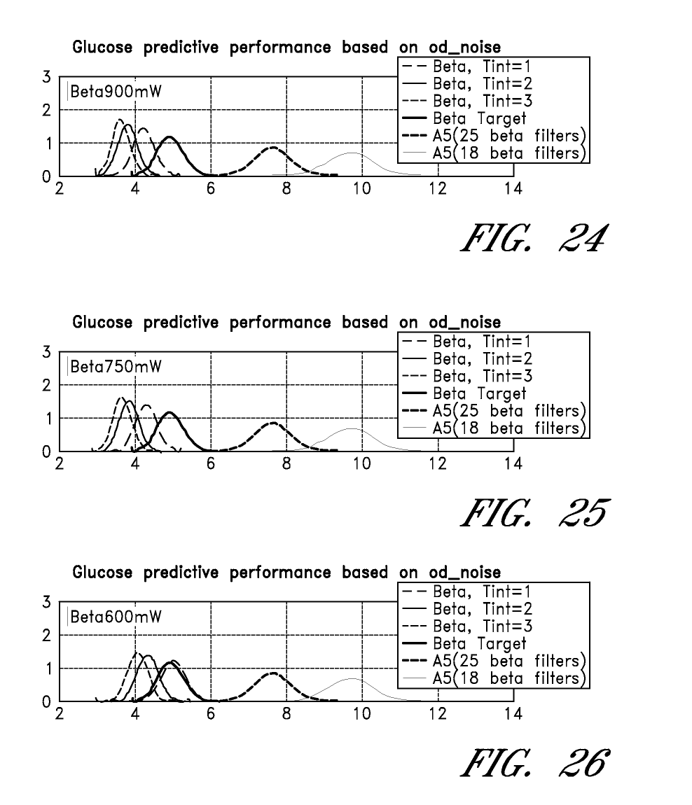

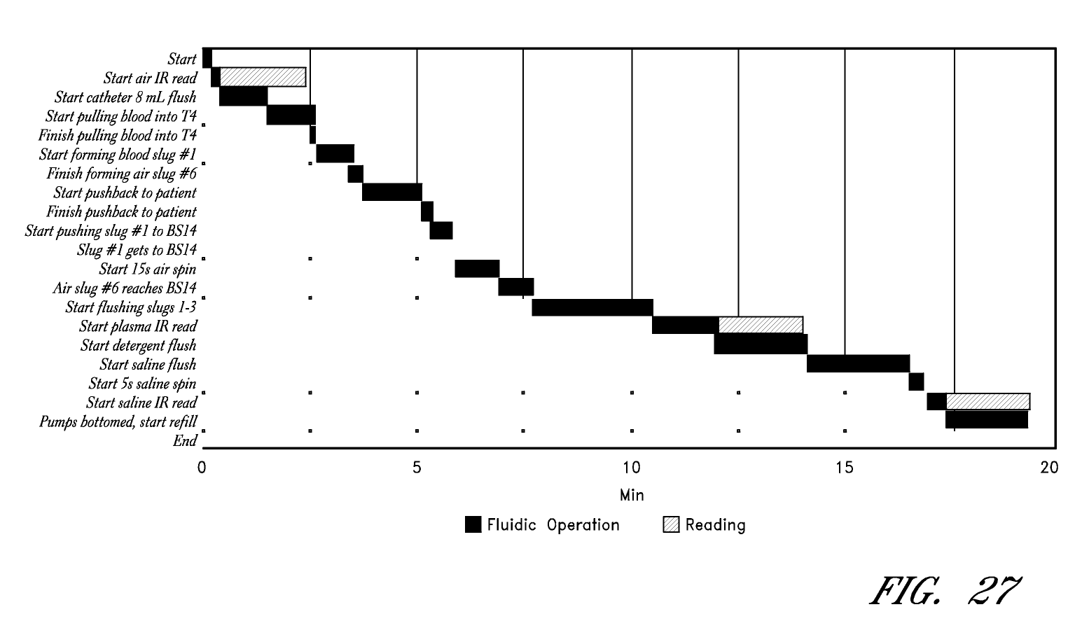

FIGS. 24, 25, and 26 illustrate the results of calculations showing the trade-off of accuracy and time for a glucose monitoring system. Specifically, FIGS. 24-26 show predictions of the performance of the spectrometer for two important variables, source power and integration time at each filter. The three graphs (labeled Beta900 mW, Beta750 mW, and Beta600 mW) represent three light source power levels (900 mW, 750 mW and 600 mW) for the optical system 1210 (see FIG. 12). The lines on the graphs (labeled Beta, Tint=1; Beta, Tint=2; and Beta, Tint=3, respectively) indicate different integration times for the spectroscopic measurements, and correspond to a total measurement time of 25 seconds, 50 seconds, and 75 seconds, respectively.

The horizontal axis in each graph of FIGS. 24-26 is standard error in mg/dL, from 2 to 14. The vertical axis has arbitrary units, and is an indicator of the number of samples at each standard error level. The number of samples is the same for each condition, and thus a sharper and higher peak is better than a lower flatter peak. In general, the higher the power and the longer the integration time, the lower the standard error. FIGS. 24-26 show that there is a trade-off between time and accuracy, and that greater accuracy can be had with a longer integration time.

### Example 4

### Cycle Time

FIG. 27 shows data for the operation time of one embodiment of the sampling system 102. In this embodiment, the total cycle is just less than 20 min. The total time per measurement can be reduced by reducing the time of fluidic operations.

Although the invention(s) presented herein have been disclosed in the context of certain preferred embodiments and examples, it will be understood by those skilled in the art that the invention(s) extend beyond the specifically disclosed embodiments to other alternative embodiments and/or uses of the invention(s) and obvious modifications and equivalents thereof. Thus, it is intended that the scope of the invention(s) herein disclosed should not be limited by the particular embodiments described above.

Methods and processes described above may be embodied in, and fully automated via, software code modules executed by one or more general purpose computers. The code modules may be stored in any type of computer-readable medium or other computer storage device. Some or all of the methods may alternatively be embodied in specialized computer hardware. The collected user feedback data (e.g., accept/rejection actions and associated metadata) can be stored in any type of computer data repository, such as relational databases and/or flat files systems.

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Further information on analyte detection systems, sample elements, algorithms and methods for computing analyte concentrations, and other related apparatus and methods can be found in U.S. Patent Application Publication No. 2003/0090649, published May 15, 2003, titled REAGENT-LESS WHOLE BLOOD GLUCOSE METER; U.S. Patent Application Publication No. 2003/0178569, published Sep. 25, 2003, titled PATHLENGTH-INDEPENDENT METHODS FOR OPTICALLY DETERMINING MATERIAL COMPOSITION; U.S. Patent Application Publication No. 2004/0019431, published Jan. 29, 2004, titled METHOD OF DETERMINING AN ANALYTE CONCENTRATION IN A SAMPLE FROM AN ABSORPTION SPECTRUM; U.S. Patent Application Publication No. 2005/0036147, published Feb. 17, 2005, titled METHOD OF DETERMINING ANALYTE CONCENTRATION IN A SAMPLE USING INFRARED TRANSMISSION DATA; and U.S. Patent Application Publication No. 2005/0038357, published on Feb. 17, 2005, titled SAMPLE ELEMENT WITH BARRIER MATERIAL. The entire contents of each of the above-mentioned publications are hereby incorporated by reference herein and are made a part of this specification.

A number of applications, publications and external documents are incorporated by reference herein. Any conflict or contradiction between a statement in the bodily text of this specification and a statement in any of the incorporated documents is to be resolved in favor of the statement in the bodily text.

Although the invention(s) presented herein have been disclosed in the context of certain preferred embodiments and examples, it will be understood by those skilled in the art that the invention(s) extend beyond the specifically disclosed embodiments to other alternative embodiments and/or uses of the invention(s) and obvious modifications and equivalents thereof. Thus, it is intended that the scope of the invention(s) herein disclosed should not be limited by the particular embodiments described above.

What is claimed is:

1. An apparatus for obtaining a measurement of a concentration of an analyte in a sample, the apparatus comprising:
a fluid line configured to provide a full-time connection to a fluid vessel;
a sample cell;
a fluid system disposed between the fluid line and the sample cell, the fluid system comprising a pump
a controller configured to cause the pump to draw the sample from the fluid vessel into the fluid system;
an analyte detection system configured to measure a concentration of an analyte in the sample; and
a reporting system configured to report the concentration of the analyte in the sample to a user of the apparatus;
wherein one or more components of the apparatus for obtaining a measurement is configured to measure the concentration of the analyte in the sample such that a time interval between drawing the sample into the fluid system and reporting the concentration of the analyte in the sample does not exceed approximately 25 minutes.

2. The apparatus of claim 1, wherein a standard error of a measurement of the concentration of the analyte in the sample obtained by the analyte detection system does not exceed about 14 milligrams per deciliter.

3. The apparatus of claim 2, wherein the standard error of a measurement does not exceed about 10 milligrams per deciliter.

4. The apparatus of claim 1, wherein the analyte comprises glucose.

5. The apparatus of claim 1, wherein the sample comprises whole blood.

6. The apparatus of claim 5, further comprising a plasma separation system configured to separate plasma from other constituents of the sample.

7. The apparatus of claim 6, wherein the plasma separation system comprises a centrifuge.

8. The apparatus of claim 1, wherein the sample cell is disposed within a centrifuge.

9. The apparatus of claim 1, wherein the time interval between drawing the sample into the fluid system and reporting the concentration of the analyte in the sample does not exceed about 15 minutes.

10. The apparatus of claim 1, wherein the fluid system is configured to share the fluid line with at least one of a continuously-operating infusion pump or a pressure transducer.

11. A method for obtaining a measurement of a concentration of an analyte in a sample, the method comprising:
priming at least a portion of an extracorporeal fluid system with a saline solution;
drawing a sample from a fluid source into the fluid system;
returning at least some of the sample to the fluid source;
separating the sample into a plurality of constituent parts;
analyzing at least one of the plurality of constituent parts of the sample to obtain a measurement of the concentration of the analyte;
flushing at least a portion of the fluid system with a saline solution; and
reporting the measurement of the concentration of the analyte within about 25 minutes of drawing the sample.

12. The method of claim 11, further comprising drawing an air slug into the fluid system.

13. The method of claim 11, wherein analyzing at least one of the plurality of constituent parts of the sample to obtain the measurement of the concentration of the analyte comprises obtaining a measurement having a standard error of not more than 14 milligrams per deciliter.

14. The method of claim 11, wherein reporting the measurement of the concentration of the analyte comprises displaying the measurement of the concentration of the analyte on a display.

15. The method of claim 11, wherein reporting the measurement of the concentration of the analyte within about 25 minutes of drawing the sample comprises reporting the measurement of the concentration of the analyte within about 10 minutes of drawing the sample.

16. The method of claim 11, wherein drawing a sample from a fluid source into the fluid system comprises drawing whole blood from a blood vessel.

17. The method of claim 16, wherein drawing whole blood from a blood vessel comprises drawing from a direct connection to one selected from the group consisting of an arterial catheter, a central venous catheter, and a peripheral venous catheter.

18. The method of claim 11, wherein analyzing at least one of the plurality of constituent parts of the sample to obtain a measurement of the concentration of the analyte comprises obtaining a measurement of the concentration of glucose in the sample.

19. The method of claim 11, wherein analyzing at least one of the plurality of constituent parts of the sample to obtain a measurement of the concentration of the analyte comprises detecting a plurality of absorption spectra in the mid-infrared range.

20. The method of claim 11, wherein returning at least some of the sample to the fluid source comprises returning substantially all but about 40 microliters or less of the sample.

21. The method of claim 11, wherein analyzing at least one of the plurality of constituent parts of the sample to obtain a measurement of the concentration of the analyte comprises using a pre-collected spectrum of a pure interfering substance to reduce the effect of the interfering substance on the measurement.
